(19) United States
(12) Patent Application Publication
(10) Pub. No.: US 2025/0166227 A1
(43) Pub. Date: May 22, 2025

(54) METHOD FOR IMAGING BRAIN FIBRE TRACTS

(71) Applicant: UCL Business Ltd, London (GB)

(72) Inventors: Fiona YOUNG, London (GB); Kristian AQUALINA, Loughton (GB); Chris CLARK, Dorking (GB); Jonathan CLAYDEN, Hitchin (GB)

(21) Appl. No.: 18/854,805

(22) PCT Filed: Apr. 13, 2023
(86) PCT No.: PCT/GB2023/050990

§ 371 Date: Oct. 7, 2024

(30) Foreign Application Priority Data
Apr. 13, 2022 (GB) 2205480.3

(51) Int. Cl. G06T 7/73; A61B 5/00; A61B 5/055; G06T 7/33

(52) U.S. Cl. CPC G06T 7/75; A61B 5/0042; A61B 5/055; G06T 7/344; G06T 2207/10088; G06T 2207/30016; G06T 2207/30096

(57) ABSTRACT

Embodiments of the present techniques provide a method for imaging specific brain fibre tracts, using an image of a subject's brain and an atlas indicating an expected location and orientation of the brain fibre tract. Advantageously, the present techniques enable brain fibre tracts to be imaged/visualised quickly, which makes it suitable for pre-surgical planning, surgical navigation, and intra-operative imaging.

### FIELD

The present techniques generally relate to imaging brain fibre tracts. In particular, the present techniques provide a method for using an atlas which indicates an expected location and expected orientation of a specific brain fibre tract in a structurally-normal brain, to quickly and efficiently determine the actual location and orientation of the specific brain fibre tract in an individual subject's brain.

### BACKGROUND

Neurosurgery carries risks to healthy brain structures, including neuron fibre bundles called white matter tracts, injury to which can cause disruption to such important functions as movement, vision and speech. The spatial relationship between neurosurgical targets and adjacent white matter tracts can be determined preoperatively from diffusion magnetic resonance imaging (dMRI). However, the information in preoperative images becomes inaccurate as the spatial relationships change over the course of surgery (brain shift).

Intraoperative dMRI offers a means for imaging fibre tracts after brain shift has invalidated preoperative imaging. However, the unique challenges of intraoperative imaging, which include strict time constraints on image acquisition and post-processing, and on the availability of specialist operators and computing equipment, mean that standard preoperative image processing techniques don't translate easily to the intraoperative environment.

The current clinical standard for reconstructing tracts from dMRI data preoperatively is streamline tractography (hereafter referred to as “tractography”), in which fibre tracking algorithms generate virtual fibres from fibre orientations modelled from dMRI data. However, there remains a notable gap between advanced tractography and tract segmentation methods widely used in dMRI research and those methods that remain commonplace in clinical practice, despite clear evidence of the accuracy and reliability drawbacks characteristic of the latter more outdated tractography techniques. A major factor behind this adoption delay is likely convenience: implementation of streamline tractography can be time-consuming, and obtaining accurate results in the presence of tumours is difficult. Generating reconstructions of specific tracts requires (usually manual) placement of anatomical regions of interest, as well as manual post-processing to remove spurious streamlines. In addition, tractography has poor reproducibility, with results depending on numerous factors. All considered, it is perhaps unsurprising that there is hesitancy within the neurosurgical community to adopt intraoperative tractography, even as interest in intraoperative imaging (such as intraoperative MRI) and tractography for surgical planning and navigation grows.

The applicant has therefore identified the need for an improved technique for imaging brain fibre tracts.

### SUMMARY

The limitations of tractography-based tract segmentation, particularly in the context of intraoperative imaging, motivate an alternative, direct segmentation method. Starting with raw or minimally pre-processed diffusion MRI, dMRI, data, one objective of the present techniques is to obtain a likelihood map of tract location, given prior information of orientation and spatial extent. Such an approach should bypass the more involved steps of current tractography pipelines, cutting back on time, computation resources and manual work.

In a first approach of the present techniques, there is provided a computer-implemented method for imaging brain fibre tracts of a subject, the method comprising: obtaining at least one tract-specific atlas of a brain, the atlas comprising a plurality of voxels indicating an expected location and a distribution of expected voxel-wise orientations of a specific brain fibre tract in a brain; obtaining a diffusion magnetic resonance imaging, dMRI, image of a brain of the subject; comparing the at least one obtained atlas with the obtained image to determine whether a brain fibre tract in the brain of the subject overlaps with the brain fibre tract of the atlas; and generating, using the comparing, a modified image of the brain of the subject showing a location of the specific brain fibre tract in the brain of the subject.

In some cases, each tract-specific atlas may indicate location and orientation information for one specific tract. As explained below, two or more tract-specific atlases may be used to identify particular brain fibre tracts in the brain of the subject, particularly when the brain fibre tracts cross (i.e. are co-located but have different orientations). In other cases, each tract-specific atlas may indicate location and orientation information for multiple specific tracts. In these cases, a single tract-specific atlas may be used to identify particular brain fibre tracts in the brain of the subject, particularly when the brain fibre tracts cross. Such atlases are still tract-specific, as they contain information about multiple, individual specific tracts, rather than, for example, averaged information about a group of tracts (which would not be useful to distinguish tracts that are co-located). Such atlases may be created by concatenating individual tract-specific atlases.

Advantageously, the present techniques enable the location and orientation of specific brain tracts to be visualised quickly and without the need for experts who can perform tractography. Since the tract-specific atlas of the present techniques indicates the likelihood of a voxel containing a specific brain fibre tract, each voxel contains information on the location and orientation of specific, individual brain fibre tracts that are expected to be in that voxel. In other words, if multiple brain fibre tracts are likely to be present in a voxel, multiple tract-specific atlases of the present techniques may be used, where each tract-specific atlas indicates orientation information for a specific brain fibre tract in that voxel. That is, the present techniques do not attempt to represent an average direction of all fibre tracts passing through a voxel in the atlas. Thus, the present techniques are more specific and discerning for modelling specific fibre tracts than existing techniques, in particular when identifying crossings between two fibres.

As mentioned above, tractography remains a technique used in research organisations. Less-advanced tractography may be used by clinicians or surgeons, but a drawback of using these less-advanced techniques is the under-representation of tracts. Sometimes tractography is only used by clinicians or surgeons pre-operatively and not intraoperatively. Furthermore, advanced, research-based tractography (such as probabilistic tractography that uses multi-fibre models) requires experts to perform tractography, which makes the technique difficult to implement in resource-limited environments (i.e. where the skilled labour does not exist). Further still, the technique can be quite time-consuming, which can significantly impact the usability of the technique during surgery. Thus, the present techniques provide a way to image brain fibre tracts without requiring experts and without requiring too much time. The imaging method of the present techniques may take only a few minutes (depending on computer processor capability and how many tracts are to be imaged), for example, which means it is more suitable as an inter-operative technique than state-of-the-art tractography.

The present imaging method may advantageously be used pre-surgery or pre-diagnosis. This may enable surgeons to plan their surgeries and navigate during a surgery. The surgical planning may include entry points into the brain and how to manoeuvre within the brain without contacting or damaging the brain fibre tract. This may be useful when surgeons are planning to implant a device within the brain to perform deep brain stimulation, such as for the treatment of Parkinson's disease. Where a brain tumour exists, the present imaging method may advantageously enable the surgeon to see how the presence of the tumour has impacted the location of the brain fibre tracts, which again impacts the planning of their surgeries and navigation during surgery (to biopsy or remove the tumour, for example). It will be understood that the term “tumour” is used herein to mean any space-occupying lesion.

A substantial challenge to tract segmentation in clinical images is the appropriate navigation of space-occupying lesions that may displace and distort the tract away from expectations. This is where existing tractography-free segmentation approaches struggle to produce satisfactory results, and where the present techniques aim to success by incorporating patient-specific lesion deformation modelling. Thus, the present imaging method may advantageously be used during surgery (i.e. intra-operatively). This may enable surgeons to quickly determine the location of a brain fibre tract during the surgery, in combination with intraoperative MRI scans (e.g. DTI diffusion weight scans, and a structural scan). This is useful if, for example, a surgeon has removed a section of a tumour, which has caused further brain shift—the previously shifted brain fibre tracts may be pushed into a new location or may move back into the location previously occupied by the section of the tumour. The speed of the present technique makes it particularly suitable to intra-operative use.

Thus, the step of obtaining an image of a brain of the subject may comprise obtaining an image of the brain pre-surgery and/or during surgery.

The image of a brain of the subject may be formed of or comprise voxels. The method may further comprise: modelling, using the obtained dMRI image, an orientation distribution of at least one brain fibre tract in each voxel of the image. The modelling may comprise using any one of the following techniques to determine an orientation distribution of at least one brain fibre tract in each voxel of the image: constrained spherical deconvolution; a multi-compartment model (such as, a multi-tensor model or a ball-and-stick model); and a multi-fibre model. It will be understood that these are non-exhaustive and non-limiting examples of possible models/techniques that could be used to determine orientation distribution.

The atlas is a voxel-wise fibre orientation atlas. The image of a brain of the subject may also be formed of or comprise voxels. Thus, the step of comparing the at least one obtained atlas with the obtained image may comprise: comparing each voxel of the at least one obtained atlas with each voxel of the obtained image.

Each voxel of the at least one obtained atlas comprises a distribution of expected orientations of a specific brain fibre tract that is expected to be located in the voxel. Comparing each voxel may comprise: obtaining a measure per voxel of how closely an orientation distribution of a brain fibre tract in each voxel of the image overlaps with the distribution of expected orientations of the specific brain fibre tract.

As noted above, in some cases, obtaining at least one tract-specific atlas of a brain may comprise obtaining at least two tract-specific atlases. In such cases, when the at least two atlases indicate that two or more brain fibre tracts are likely to be located in a particular voxel, comparing each voxel may comprise: obtaining a measure per voxel of how closely an orientation distribution of a brain fibre tract in each voxel of the image overlaps with each distribution of expected orientations of the brain fibre tracts in the at least two atlases; and determining which one of the two or more brain fibre tracts is present in the voxel of the obtained image based on the obtained measure.

Alternatively, in some cases, obtaining at least one tract-specific atlas of a brain may comprise obtaining a tract-specific atlas that contains information about multiple specific tracts. In such cases, when this multi-tract atlas indicates that two or more brain fibre tracts are likely to be located in a particular voxel, comparing each voxel may comprise: obtaining a measure per voxel of how closely an orientation distribution of a brain fibre tract in each voxel of the image overlaps with each distribution of expected orientations of the two or more brain fibre tracts in the atlas; and determining whether one or more of the two or more brain fibre tracts is present in the voxel of the obtained image based on the obtained measure. In some cases, a single tract may be determined to be present in the voxel of the obtained image, and in other cases multiple tracts (e.g. overlapping tracts) may be determined to be present.

The atlas may be represented by a first spherical distribution function, and the obtained image may be represented by a second spherical distribution function. The first and second spherical distribution functions may be spherical harmonic distribution functions. Thus, the step of comparing may comprise calculating an integral of a product of the first and second functions. The calculating may comprise calculating a voxel-wise integral of a product of the first and second functions.

Using a spherical distribution function representation enables the use of constrained spherical deconvolution (CSD), which allows for more accurate identification of a crossing between two fibre tracts. Additionally, or alternatively, other multi-fibre models may be used to achieve this increase in accuracy. Additionally, or alternatively, the first and second spherical distribution functions may be compared using a different similarity metric, distance metric or similarity measure, such as the Kullback-Leibler divergence metric or other f-divergence. In this case, the calculating may comprise calculating a Kullback-Leibler divergence metric using the first and second functions. It will be understood that these are non-exhaustive and non-limiting examples of metrics that may be used to compare two spherical distribution functions.

Generating a modified image may comprise outputting an image representing a result of the calculating.

In some cases, the step of obtaining the at least one atlas may comprise obtaining at least one atlas indicating an expected location and an expected orientation of a specific brain fibre tract in a structurally normal brain (i.e. tumour- or lesion-free brain).

Alternatively, the step of obtaining the at least one atlas may comprise obtaining at least one atlas that has been pre-deformed, the pre-deformed atlas indicating an expected location and an expected orientation of a specific brain fibre tract in a brain containing a tumour. In this case, the pre-deformed atlas may be generated by transforming an atlas indicating an expected location and an expected orientation of a specific brain fibre tract in a structurally normal brain, using a tumour model that defines how brain fibre tracts are, or more broadly how brain tissue is, displaced by tumours. This is explained in more detail below.

In a second approach of the present techniques, there is provided a computer-implemented method for generating a tract-specific atlas of a structurally normal brain for use in brain fibre tract imaging, the method comprising: obtaining a plurality of images of structurally normal brains of multiple subjects; extracting, from each image, spatial location and orientation information of at least one brain fibre tract in the brain; and generating, using the extracted spatial location and orientation information, an atlas comprising a plurality of voxels indicating an expected location and a distribution of expected orientations of at least one specific brain fibre tract.

Generating an atlas may comprise: determining, using the extracted spatial location information from the plurality of images, a likelihood of a specific brain fibre tract being located in a particular voxel.

Generating an atlas may comprise: determining, using the extracted orientation information from the plurality of images, a distribution of orientations of a specific brain fibre tract in a particular voxel.

When two or more brain fibre tracts are likely to be located in a particular voxel, generating an atlas may comprise: including, in the particular voxel of the atlas, the distribution of expected orientations of each of the two or more brain fibre tracts. In this way, a multi-tract atlas may be generated, which includes location and orientation information for multiple specific tracts individually. Such a multi-tract atlas may be generated by concatenating separate atlases for each specific tract.

Obtaining a plurality of images of brains may comprise obtaining images acquired from a high angular resolution diffusion imaging, HARDI, process.

In a third approach of the present techniques, there is provided a computer-implemented method for generating a pre-deformed atlas for use in brain fibre tract imaging, the method comprising: obtaining information about a subject having a brain tumour; obtaining an atlas of a specific brain fibre tract of interest, the atlas indicating an expected location and an expected orientation of the specific brain fibre tract in a structurally normal brain; and transforming the atlas, using the obtained information and a tumour model that defines how brain fibre tracts are displaced by tumours, to generate a pre-deformed atlas. The atlas of a specific brain fibre tract of interest may be a single atlas that contains location and orientation information about a single specific brain fibre tract, or may be a single atlas that contains location and orientation information about multiple specific brain fibre tracts separately (rather than in some averaged way).

Advantageously, by having a pre-deformed atlas that has been generated using a tumour model, it is possible to quickly determine the expected location and orientation of a brain fibre tract in a brain containing a tumour. As mentioned above, this can enable a surgeon or clinician to perform surgical planning and navigation, both pre-surgery and during surgery.

The step of obtaining information about a subject having a brain tumour may comprise obtaining information on a location of the brain tumour. For example, the information on a location of the brain tumour may be acquired from an image of the brain of the subject. The image of the brain may be a dMRI image, or an image obtained from a structural scan (e.g. a T1 or T2 weighted MRI).

The tumour model may be a radial tumour expansion model.

The atlas may comprise a plurality of voxels, and transforming the atlas may comprise: defining, for each voxel, a distance to a centre of mass of a tumour; and applying, to each voxel, a function which defines an amount by which each voxel is displaced as being dependent on the distance from the voxel to a centre of mass of a tumour, a distance from the centre of mass to a surface of the brain, and a distance from the centre of mass to a surface of the tumour.

Applying a function may comprise applying any one of the following: an exponentially decaying function; a polynomial function; a probability density function of a logistic distribution or of a hyperbolic secant distribution function; a damped oscillator function; and a linear function. It will be understood that these are non-exhaustive and non-limiting examples of functions that may be used to determine the impact of a tumour on the voxels.

The tumour model may model how fibre tracts are displaced by infiltrating and/or non-infiltrating tumours.

In a fourth approach of the present techniques, there is provided a pre-deformed atlas for use in brain fibre tract imaging generated using the method of the second approach.

In a fifth approach of the present techniques, there is provided a tract-specific atlas of a structurally normal brain for use in brain fibre tract imaging generated using the method of the third approach.

In a sixth approach of the present techniques, there is provided an image processing system comprising: an image capture device which is configured to capture an image; an image processor which is configured to receive an image from the image capture device and carry out the imaging method described herein; and a user interface which is configured to display an output result generated by the image processor.

In a related approach of the present techniques, there is provided a non-transitory data carrier carrying processor control code to implement any of the methods, processes and techniques described herein.

As will be appreciated by one skilled in the art, the present techniques may be embodied as a system, method or computer program product. Accordingly, present techniques may take the form of an entirely hardware embodiment, an entirely software embodiment, or an embodiment combining software and hardware aspects.

Furthermore, the present techniques may take the form of a computer program product embodied in a computer readable medium having computer readable program code embodied thereon. The computer readable medium may be a computer readable signal medium or a computer readable storage medium. A computer readable medium may be, for example, but is not limited to, an electronic, magnetic, optical, electromagnetic, infrared, or semiconductor system, apparatus, or device, or any suitable combination of the foregoing.

Computer program code for carrying out operations of the present techniques may be written in any combination of one or more programming languages, including object oriented programming languages and conventional procedural programming languages. Code components may be embodied as procedures, methods or the like, and may comprise sub-components which may take the form of instructions or sequences of instructions at any of the levels of abstraction, from the direct machine instructions of a native instruction set to high-level compiled or interpreted language constructs.

Embodiments of the present techniques also provide a non-transitory data carrier carrying code which, when implemented on a processor, causes the processor to carry out any of the methods described herein.

The techniques further provide processor control code to implement the above-described methods, for example on a general purpose computer system or on a digital signal processor (DSP). The techniques also provide a carrier carrying processor control code to, when running, implement any of the above methods, in particular on a non-transitory data carrier. The code may be provided on a carrier such as a disk, a microprocessor, CD- or DVD-ROM, programmed memory such as non-volatile memory (e.g. Flash) or read-only memory (firmware), or on a data carrier such as an optical or electrical signal carrier. Code (and/or data) to implement embodiments of the techniques described herein may comprise source, object or executable code in a conventional programming language (interpreted or compiled) such as C, or assembly code, code for setting up or controlling an ASIC (Application Specific Integrated Circuit) or FPGA (Field Programmable Gate Array), or code for a hardware description language such as Verilog® or VHDL (Very high speed integrated circuit Hardware Description Language). As the skilled person will appreciate, such code and/or data may be distributed between a plurality of coupled components in communication with one another. The techniques may comprise a controller which includes a microprocessor, working memory and program memory coupled to one or more of the components of the system.

It will also be clear to one of skill in the art that all or part of a logical method according to embodiments of the present techniques may suitably be embodied in a logic apparatus comprising logic elements to perform the steps of the above-described methods, and that such logic elements may comprise components such as logic gates in, for example a programmable logic array or application-specific integrated circuit. Such a logic arrangement may further be embodied in enabling elements for temporarily or permanently establishing logic structures in such an array or circuit using, for example, a virtual hardware descriptor language, which may be stored and transmitted using fixed or transmittable carrier media.

In an embodiment, the present techniques may be implemented using multiple processors or control circuits. The present techniques may be adapted to run on, or integrated into, the operating system of an apparatus.

In an embodiment, the present techniques may be realised in the form of a data carrier having functional data thereon, said functional data comprising functional computer data structures to, when loaded into a computer system or network and operated upon thereby, enable said computer system to perform all the steps of the above-described method.

### BRIEF DESCRIPTION OF THE DRAWINGS

Implementations of the present techniques will now be described, by way of example only, with reference to the accompanying drawings, in which:

FIG. 1 is a flowchart of example steps for imaging brain fibre tracts of a subject;

FIG. 2 shows the two components of the atlas (location and orientation) and how they are relevant to generate images of deformed brain fibre tracts;

FIG. 3 is a schematic diagram showing variables in the tumour model described herein;

FIG. 4 illustrates the use of the tumour model on test images, using different values for the decay parameter A;

FIG. 5 shows how a modified image of the brain of the subject showing a location and orientation of the specific brain tract is generated;

FIG. 6 shows use of the present imaging technique to generate an image of brain fibre tracts intraoperatively by adjusting a deformation model that has been obtained preoperatively;

FIG. 7 shows a comparison of different techniques for generating an image of brain fibre tracts;

FIG. 8A is a flowchart of example steps for generating a tract-specific atlas for imaging brain fibre tracts of a subject;

FIG. 8B is a flowchart of example steps for generating a pre-deformed atlas for imaging brain fibre tracts of a subject;

FIG. 9 is a diagram of a system for image processing;

FIGS. 10a to 10d show qualitative results obtained using the techniques of FIG. 1;

FIG. 11 is a table showing all pairwise comparisons for the HCP dataset;

FIG. 12a to 12c give an indication of how five different segmentations, including the present technique, compare with each other, across all HCP dataset subjects; and

FIG. 13 compares each studied method against the reference streamline bundles in the TractoInferno dataset.

### DETAILED DESCRIPTION OF THE DRAWINGS

Broadly speaking, embodiments of the present techniques provide a method for imaging specific brain fibre tracts, using an image of a subject's brain and an atlas indicating an expected location and orientation of the brain fibre tract. Advantageously, the present techniques enable brain fibre tracts to be imaged/visualised quickly, which makes it suitable for pre-surgical planning, surgical navigation, and intra-operative imaging.

Currently intraoperative streamline tractography is mostly limited to the tools available in commercial navigation software. For example, the Plan FibreTracking module in Brainlab surgical navigation tools (Brainlab, Feldkirchen, Germany) uses FACT (fibre assignment by continuous tracking), a deterministic, diffusion tensor derived tracking algorithm first proposed in 1998. It is not appropriate to simply update the tractography techniques implemented in these commercial tools. In general, the ill-posed nature of tractography results in a trade-off between sensitivity and specificity, with those methods common in clinical use generally exhibiting low spatial coverage of tracts (low sensitivity), while state-of-the-art tractography is afflicted by high numbers of spurious streamlines (low specificity), which could unhelpfully obscure intraoperative navigation. Instead, there is a need for alternative tract segmentation methods that do not directly utilise streamline tractography. For example, TractSeg, a deep neural network model for direct tract segmentation, has been proposed for use in neurosurgical patients. However, TractSeg does not incorporate any explicit handling of lesion mass-effects, leading to partially incomplete segmentations in some cases.

The present techniques provide an atlas-based method, dubbed “tractfinder”. In some cases, the present techniques comprise patient-specific lesion deformation modelling.

One difficulty of using atlas-based segmentation methods in clinical subjects is that of anatomical non-correspondence between subject and template images caused by space-occupying lesions. Deformable registration alone is often insufficient for handling this mismatch, and so using tumour growth models to simulate the deformation in the atlas prior to registration is the commonly preferred approach. Numerous previously proposed tumour deformation models aim to achieve highly accurate modelling of tumour growth dynamics and the effects on surrounding tissues, by taking into account elastic tissue properties and microscopic tumour growth modelling. The resulting algorithms are mathematically complex, require optimisation of tumour parameters through problem inversion or by other means and take anywhere between 1 and 36 hours to run, even on high performance computing setups.

Given the time constraints of intraoperative imaging and the practical constraints of the computing capacity which can reasonably be assumed to be available in an operating room, the present techniques aim to achieve an estimate of tract displacement with low computational complexity. The first component of tractfinder, the tract orientation atlas, provides a degree of spatial tolerance that alleviates the need for voxel-perfect registration and deformation, allowing the implementation of a minimal deformation algorithm. A tract segmentation is then derived from the overlap between the deformed atlas and fibre orientation information in the target image.

The novel contributions of this work are explicit handling of large-scale deformations and an automated pipeline that can produce results within a few minutes. The pipeline can be run fully automatically with minimal to no user input, depending on the particularities of an individual case (such as lesion mass effect and extent of resection). Tractfinder has been developed specifically for intraoperative imaging, but is equally applicable to any diffusion MRI data.

Background information can be found in EP2141506A2, which describes a method for identifying fibre tracts using magnetic resonance data and a fibre tract atlas. The fibre tract atlas is used to find a probability that a voxel in the magnetic resonance data represents a fibre tract, using a diffusion vector generated for the voxel and information on the orientations of the fibre tract from the fibre tract atlas. However, in EP2141506A2, orientation information is encoded by averaging diffusion tensor principal eigenvectors across subjects. This means that where multiple fibre tracts cross, the atlas indicates an average orientation for the multiple fibres, which does not accurately reflect the orientation of any of the crossing fibre tracts individually. In contrast, and as explained below, in the present techniques take a tract-specific approach to orientation. Furthermore, in EP2141506A2, spatial probability is given by the averaged, normalised track density values from individual deterministic streamline tractography

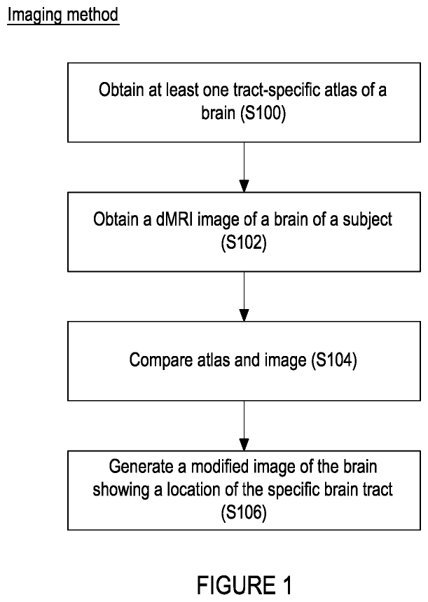

FIG. 1 is a flowchart of example steps for imaging brain fibre tracts of a subject. The method depicted in FIG. 1 comprises: obtaining at least one atlas of a brain, the atlas comprising a plurality of voxels indicating an expected location and an expected voxel-wise orientation of a specific brain fibre tract in a brain (step S100).

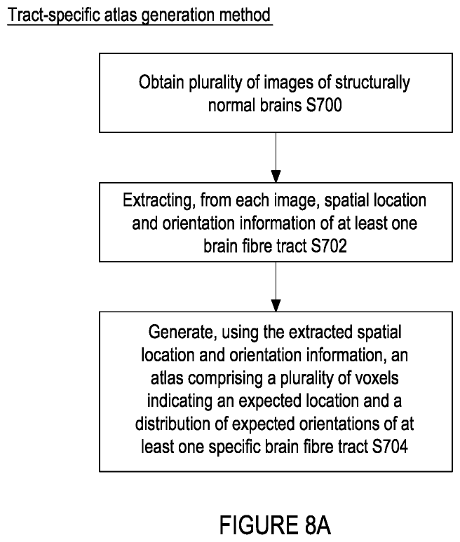

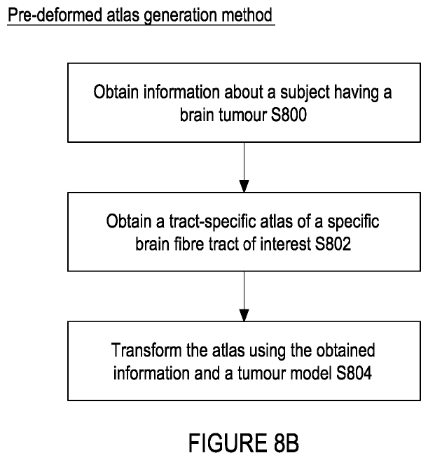

Step S100 may comprise obtaining at least one tract-specific atlas indicating an expected location and a distribution of expected orientations of a specific brain fibre tract in a structurally normal brain. This type of atlas may be obtained when, for example, a surgeon is planning a surgery to implant a medical device within the brain. FIG. 8A shows how such an atlas may be generated.

Alternatively, step S100 may comprise obtaining at least one atlas that has been pre-deformed, the pre-deformed atlas indicating an expected location and an expected orientation of a specific brain fibre tract in a brain containing a tumour. In this case, the pre-deformed atlas may be generated by transforming an atlas indicating an expected location and an expected orientation of a specific brain fibre tract in a structurally normal brain, using a tumour model that defines how brain fibre tracts are displaced by tumours. This is explained in more detail below. This type of atlas may be obtained when, for example, a surgeon is planning a surgery to biopsy or remove a tumour, or during such a surgery.

The method of FIG. 1 comprises obtaining an image of a brain of the subject (step S102). Step S102 may comprise obtaining a diffusion magnetic resonance imaging, dMRI, image of the brain pre-surgery and/or during surgery.

The image of a brain of the subject may be formed of or comprise voxels. The method may further comprise: modelling, using the obtained dMRI image, an orientation distribution of at least one brain fibre tract in each voxel of the image. The modelling may comprise using constrained spherical deconvolution to determine an orientation distribution of at least one brain fibre tract in each voxel of the image.

The method of FIG. 1 comprises comparing the at least one obtained atlas with the obtained image to determine whether the brain fibre tract in the brain of the subject overlaps with the brain fibre tract of the atlas (step S104). The image of a brain of the subject may also be formed of or comprise voxels. Thus, the step (S104) of comparing the at least one obtained atlas with the obtained image may comprise: comparing each voxel of the at least one obtained atlas with each voxel of the obtained image.

Each voxel of the at least one obtained atlas comprises a distribution of expected orientations of a specific brain fibre tract that is expected to be located in the voxel. Comparing each voxel may comprise: obtaining a measure per voxel of how closely an orientation distribution of a brain fibre tract in each voxel of the image overlaps with the distribution of expected orientations of the specific brain fibre tract.

As noted above, in some cases, obtaining at least one tract-specific atlas of a brain may comprise obtaining at least two tract-specific atlases. In such cases, when the at least two atlases indicate that two or more brain fibre tracts are likely to be located in a particular voxel, comparing each voxel may comprise: obtaining a measure per voxel of how closely an orientation distribution of a brain fibre tract in each voxel of the image overlaps with each distribution of expected orientations of the brain fibre tracts in the at least two atlases; and determining which one of the two or more brain fibre tracts is present in the voxel of the obtained image based on the obtained measure.

Alternatively, in some cases, obtaining at least one tract-specific atlas of a brain may comprise obtaining a tract-specific atlas that contains information about multiple specific tracts. In such cases, when this multi-tract atlas indicates that two or more brain fibre tracts are likely to be located in a particular voxel, comparing each voxel may comprise: obtaining a measure per voxel of how closely an orientation distribution of a brain fibre tract in each voxel of the image overlaps with each distribution of expected orientations of the two or more brain fibre tracts in the atlas; and determining whether one or more of the two or more brain fibre tracts is present in the voxel of the obtained image based on the obtained measure.

The atlas may be represented by a first spherical distribution function, and the obtained image may be represented by a second spherical distribution function. Thus, the step (S104) of comparing may comprise calculating an integral of a product of the first and second functions. The first and second functions may be spherical harmonic distribution functions. The calculating may comprise calculating a voxel-wise integral of a product of the first and second functions.

Using a spherical distribution function representation enables the use of constrained spherical deconvolution (CSD), which allows for more accurate identification of a crossing between two fibre tracts. Additionally, or alternatively, other multi-fibre models may be used to achieve this increase in accuracy.

Additionally, or alternatively, the first and second spherical distribution functions may be compared using a different metric, such as the Kullback-Leibler divergence metric:

$$K L ( T , F ) = {\int }_{Ω} T ( θ , ϕ ) ln ( \frac{T ( θ , ϕ )}{F ( θ , ϕ )} ) d θ d ϕ$$

where T(θ, ϕ) is the spherical distribution function representing the atlas (in a given voxel) and F(θ, ϕ) is the spherical distribution function representing the fibre tracts present in the same voxel in the obtained image, In is the natural logarithm and ¢_{Ω} . . . is the integral overall spherical angles. (For comparison: the equation for the integral product would be: IP(T,F)=∫_{Ω}T(θ, ϕ)F(θ, ϕ)dθdϕ). In this case, the calculating may comprise calculating a Kullback-Leibler divergence metric using the first and second functions.

The method of FIG. 1 comprises generating, using the comparing, a modified image of the brain of the subject showing a location of the specific brain fibre tract in the brain of the subject (step S106). Step S106 may comprise outputting an image representing a result of the calculating.

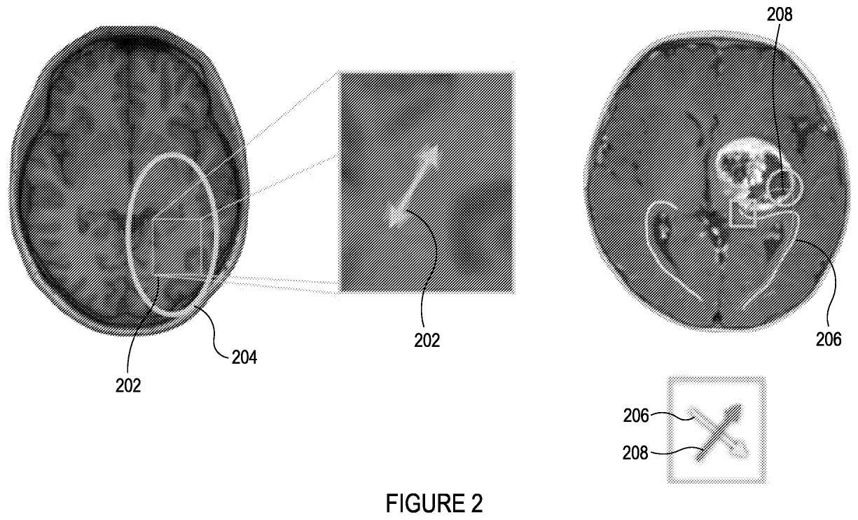

As mentioned above, the method shown in FIG. 1 may be applied to images of structurally normal brains (i.e. tumour- or lesion-free brains), and/or to images of brains having a tumour. In the latter case, the atlas that is obtained at step S100 is the pre-deformed atlas. FIG. 2 shows two components of the atlas (location and orientation) and how they are relevant to generate images of displaced brain fibre tracts (i.e. brain fibre tracts that have been displaced by space-occupying tumours). The tractfinder pipeline consists of three main components:
1. The first component, the tract atlas, illustrated in FIG. 2, acts as a first guess of a tract's spatial location 204 and orientation 202. The tract atlas incorporates known knowledge about tracts in a way that is similar to the use of regions of interest in tractography. As shown in FIG. 2, the expected location and/or orientation 208 and actual location and/or orientation 206 of a tract may be very different, particularly if the brain contains a tumour that shifts the location of the tract. (The crossing arrows in the box depict different orientations—i.e. the difference between the expected and actual orientation—while the circle shows the difference between the expected and actual location).
2. The second component is tumour deformation modelling of the atlas. This corrects for the displacement of tracts by space-occupying lesions. Minimal adjustment to precomputed deformations can account for intraoperative brain shift.
3. The third component is the generation of an image of a subject's brain, using the deformed tract atlas and target dMRI fibre orientation data, which shows a likelihood map for the tract.

Throughout this document, wherever an orientation distribution is mentioned, it will be understood that all such orientation distributions are represented in spherical harmonic (SH) basis. (However, as mentioned above, other techniques may be used to determine orientation distribution, and some of these may not be represented in a SH basis). Using this framework, a distribution is parameterised by its SH coefficients up to a maximum order set to lₘₐₓ=8 unless otherwise specified.

### Tract Orientation Atlas

The purpose of the tract atlas is to capture and store prior anatomical knowledge of a given tract, including its typical location and orientation across subjects. While this is hereinafter referred to simply as tract orientation atlas, and this section will focus on the orientation component, each final tract atlas incorporates both orientational and spatial information.

The objective is to create a map in template space capturing, at each location, the range of possible orientations the tract can take on as a single spherical distribution. A narrow distribution may be found where the tract's orientation is highly consistent across all subjects, whereas a more spread-out distribution would reflect a wider range of possible orientations, which may be seen in regions of fanning or sharp turning.

As mentioned above with reference to FIG. 1, a separate atlas is created for each tract of interest to capture its typical orientation and location. To date, bilateral atlases have been constructed for the optic radiation and corticospinal tract and arcuate fasciculus.

To obtain such a mapping, a combination of streamline tractography and tract orientation distribution, TOD, mapping is used. While tractography has numerous limitations, it remains the standard way of segmenting white matter bundles from in vivo dMRI data, and with the right postprocessing efforts biases and errors can be at least partially corrected for. In addition, tractography uniquely enables the extraction of orientation information specific to the reconstructed bundle, which would not be possible from a binary voxel-wise segmentation.

First, the tract is reconstructed in each of a series of healthy (structurally normal) training dMRI datasets (n=16) using multi-shell multi-tissue constrained spherical deconvolution and probabilistic streamline tractography and a consistent ROI-based reconstruction protocol. The dataset that was used is: “EEG, fMRI and NODDI dataset” (Clayden and Deligianni 2020), available online at osf.io/94c5t. After initial streamline generation, each tract reconstruction undergoes further postprocessing. In each subject, the bundle of interest was reconstructed in both hemispheres using probabilistic streamline tractography with iFOD2 as described in: “Improved Probabilistic Streamlines Tractography by 2nd Order Integration over Fibre Orientation Distributions.” by Tournier, et al published in Proceedings of the International Society for Magnetic Resonance in Medicine, 18:1670. in 2010, and a consistent ROI strategy based on anatomical landmarks broadly agreed upon in prior works. Spurious streamlines are automatically filtered out by creating a track density image with 2 mm cubic voxels, thresholding at 3 streamlines, and using the resulting mask to exclude stray/erroneous streamlines. After manual filtering of biologically implausible streamlines, the reconstructions are transformed to MNI space using linear registration between the subject's T1 weighted image and the MN1152 T1 template, as described in: “Unbiased Average Age-Appropriate Atlases for Pediatric Studies.” By Fonov et al published in NeuroImage 54 (1): 313-27. https://doi.org/10.1016/j.neuroimage.2010.07.033. in 2011. Affine registration rather than non-linear, was used for this step to capture individual anatomical variation and minimise unrealistic warping of streamlines from local registration errors or overfitting. With all streamlines aggregated in MNI space, manual filtering of streamlines was performed to remove not only “volumetric false positives”, which depart from the accepted volume of the tract, but also “orientational false positives” (OFPs), which remain entirely within the tract volume but are at least in part aligned with a different, intersecting bundle. Such streamlines have little effect on any volumetric applications of the reconstruction, e.g. via a track density depiction. However, their removal is vital for the construction of the orientation atlas, which summarises the orientational distribution of streamlines on a voxel-wise basis. Filtering was performed in DSI studio (v2021_04, https://dsi-studio.labsolver.org/ by Yeh 2021), which enables the filtering of streamlines based on angle of intersection with a cutting plane.

Once in MNI space, the tract orientation distribution, TOD, is computed from the streamlines using tract orientation density imaging.

After aggregate filtering, the retained streamlines were re-separated into individual subject bundles and the TOD was computed from the individual bundles as described in: “Track orientation density imaging (TODI) and track orientation distribution (TOD) based tractography” by Dhollander et al published in NeuroImage 94, 312{336. doi:10.1016/j.neuroimage. 2013.12.047 in 2014 and implemented in MRtrix3 as described in: “MRtrix3: A fast, flexible and open software framework for medical image processing and visualization” by Tournier et al. published in NeuroImage 202, 116137. doi:10.1016/j.neuroimage.2019.116137 in 2019. TOD mapping is the generalisation of track density imaging into the angular domain, creating a 5D spatio-angular representation of streamline tracks on a voxel-wise basis. The TOD image is represented in modified spherical harmonic basis using only even orders up to a maximum order lₘₐₓ=8, meaning each image consists of 45 coefficients, denoted tⱼ, per voxel. The distribution is described by those coefficients and the modified spherical harmonic basis functions Y_{l,m} as

$$T ( θ , ϕ ) = {\sum }_{l = 0}^{{l}_{max}} {\sum }_{m = - l}^{l} {t}_{l , m} {Y}_{l , m} ( θ , ϕ ) = {\sum }_{j} {t}_{j} {Y}_{j} ( θ , ϕ )$$

Next, tract orientation distribution (TOD) mapping is used to calculate the distribution of streamline orientations within each voxel. The TOD map is normalised to unit integral on the sphere in order to remove streamline density information. The individual TOD images at this stage still contain significant density bias, with exaggerated differences in magnitude between the core bundle portions and fanning extremities owing to tractography's tendency towards early termination outside of the densest collinear tract regions. The purpose of the atlas is to capture only the likelihood of a tract's presence in any given voxel (spatial prior) and, in the case that it is present, its expected orientation (orientational prior). If the spatial prior is to be determined by considering the spatial variation of the tract between subjects, then the only information needed for each individual subject is a binary visitation map for the bundle and orientational data. Thus, to remove the streamline density component, the TOD maps for each subject are normalised as follows. The spherical integral of each SH basis function Y_{l,m} is

$${\int }_{Ω} {Y}_{l}^{m} ( θ , ϕ ) = \{ \begin{matrix}\sqrt{4 π} & if l = m = 0 \\ 0 & otherwise\end{matrix} .$$

Using the sum and constant rules of integration, the spherical integral of T(θ, ϕ) is

$${\int }_{Ω} T ( θ , ϕ ) = {t}_{0} \sqrt{4 π}$$

where t₀ is the first SH coefficient for l=m=0. Thus, to remove density information the TOD map is normalised to unit integral as

$$\tilde{T} ( θ , ϕ ) = \frac{T ( θ , ϕ )}{\sqrt{4 π} {t}_{0}}$$

After each training subject's TOD map has been normalised in MNI space, what remains contains only information about the tract's streamline orientations, and no information about the number of streamlines passing through a given voxel in the original reconstruction.

Finally, the mean over all individual training normalised TOD maps is computed to produce the final tract TOD atlas. Averaging all maps results in distributions that reflect all possible ranges of tract orientations in each voxel. The averaging step introduces a spatial probability component to the atlas. The first coefficient of the atlas will reflect the proportion of training subjects in which the tract was present in a given voxel. In this way, outlier voxels visited by only a small number of streamlines in a single subject's reconstruction will contribute less to the final atlas, and subjects with a track density of zero in a given voxel contribute nothing to that part of the atlas.

The resulting tract atlas is an average map of the tract over all training subjects, which contains both a spatial (first coefficient) and orientational component. For use in unseen target subjects, the tract atlas is linearly registered to the target image for subsequent calculations.

### Tumour Deformation Modelling

The tract orientation atlas represents the expected orientation and location of the tract for a typical healthy subject. In particular, the orientation atlas summarises the orientational distribution of streamlines on a voxel-wise basis. In order to correct for displacement of white matter tracts due to space occupying lesions, a simple radial tumour expansion model is employed.

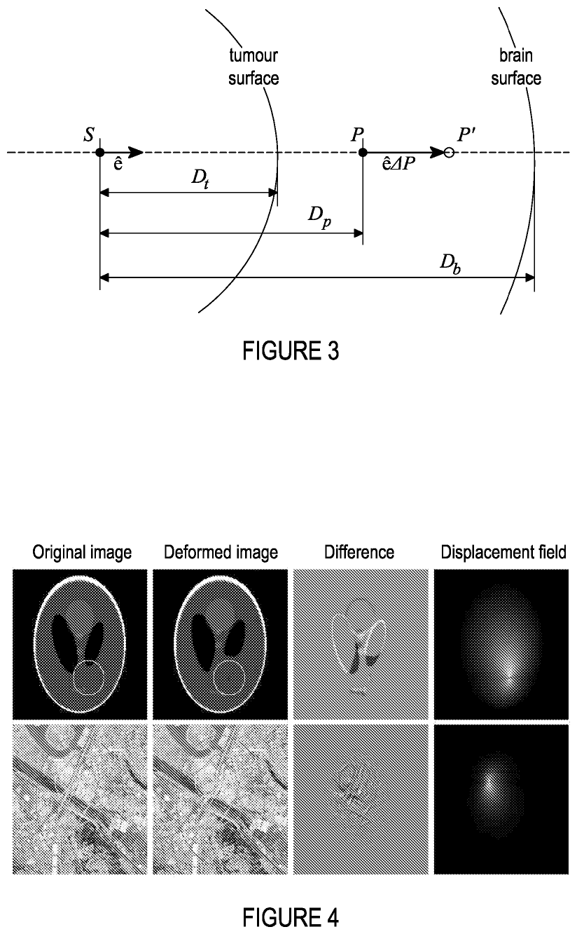

The model has been adapted from the one described by Nowinski and Belov (Nowinski, W. L. & Belov, D. Toward atlas-assisted automatic interpretation of MRI morphological brain scans in the presence of tumour. Academic Radiology 12 (8), 1049-1057 (2005)). The model inputs are the segmentations of the tumour and brain volumes. Tumour segmentations were drawn manually for this study, while brain masks are readily computed by available MRI analysis software. FIG. 3 is a schematic diagram showing variables in the tumour model described herein. In the present techniques, as shown by FIG. 3, the direction ê is defined, which is the unit vector along the line connecting a point P(x, y, z) to the tumour centre of mass, S. Along ê, further distances are also defined: Dₚ as the distance ∥\vec{SP}∥, D_{b} as the distance from S to the brain surface, and Dₜ as the distance from S to the tumour surface.

Then for a point in the original image P=(x, y, z) the transformed location in the deformed image P′=(x′, y′, z′) is

$${P}^{′} = f ( P ) = P + \hat{e} {kD}_{t} s . \qquad ( 1 )$$

An exponentially decaying function is used to model the displacement of each voxel. This choice was made in contrast to the linear relationship used in Nowinski and Belov, as it provides a better approximation to typically observed tumour displacement patterns, while remaining an easily computable, closed-form and invertible function. The amount of displacement depends exponentially on the relative distance to the tumour and brain surfaces via the following relationship:

$$k ( P ) = ( 1 - c ) {e}^{- λ \frac{{D}_{p}}{{D}_{b}}} + c , \qquad ( 2 )$$

where the normalisation constant

$$c = \frac{{e}^{- λ}}{{e}^{- λ} - 1}$$

ensures that k=1 when D_{P}=0 and k=0 when Dₚ=D_{b}. The appropriate value for the decay parameter λ will depend on the specific lesion being modelled. For example, smaller lesions (20-30 mm diameter) typically displace tissue only in their immediate surroundings, with distant tissue remaining virtually unmoved. In such cases, a higher value of λ (≥3), indicating stronger decay of deformation, would be appropriate (FIG. 1). FIG. 4 illustrates the use of the tumour model on test images, using two different values for the decay parameter λ. In the top set of images, a Shepp-Logan phantom is depicted, and λ=2, while the bottom set of images show a San Diego aerial test image, and λ=6. (These images are obtained from https://ieeexplore.ieee.org/document/6499235 and https://sipi.usc.edu/database/database.php). It can be seen from FIG. 4 that larger values of A result in more localized deformation fields, while normalization ensures deformation is always zero at the brain boundary (shown as an ellipse). The circle illustrates a simulated tumour boundary.

Equations (1) and (2) describe the deformation field in forward warp convention. To deform an image using reverse warp (“pull-back”) convention, the inverse mapping P′=f⁻¹(P) is needed, which is obtained by solving equation (1) for P:

$$P = {P}^{′} - ( {D}_{t} c - \frac{{D}_{b}}{λ} {𝒲}_{0} ( \frac{- λ {D}_{t} ( 1 - c ) {e}^{- λ ( {D}_{{p}^{′}} - {D}_{t} c ) / {D}_{b}}}{{D}_{b}} ) ) , \qquad ( 3 )$$

where W₀(y) is the principal branch of the Lambert W function, defined as the inverse function of y(x)=xe^{x} for x, y∈ [symbol] .

If the lesion is not invading the surrounding tissue but is instead fully displacing it (non-infiltrative), then under the simplified assumption that no original, healthy tissue is destroyed, λ should be set to a value that ensures that every point P within the lesion boundary is displaced to a new position P′ that is strictly outside the boundary. In other words,

$$k ( P ) = ( 1 - c ) {e}^{- λ \frac{{D}_{P}}{{D}_{b}}} + c \geq 1 - \frac{{D}_{P}}{{D}_{t}} \qquad ( 4 )$$

must hold for all P.

Given that the gradient of k is strictly decreasing and

$$g ( {D}_{P} ) = 1 - \frac{{D}_{P}}{{D}_{t}}$$

is linear, it is sufficient to set

$${{\frac{d}{dP} \vert}_{{D}_{P} = 0} k ( {D}_{P} ) = \frac{d}{dP} \vert}_{{D}_{P} = 0} g ( {D}_{P} ) . \qquad ( 5 )$$

Differentiating both functions at D_{P}=0 and solving for λ, gives

$${λ}_{max} = \frac{{D}_{b}}{{D}_{t} ( 1 - c )} .$$

Thus for strictly non-infiltrating lesions, λ≤λₘₐₓ is set as a condition to satisfy equation (4), where λₘₐₓ is used as the default value if none is specified. Note that λₘₐₓ varies throughout the brain, as it depends on the relative distances to brain and tumour surfaces for each specific P.

The tumour deformation model is implemented in Python, and full execution takes on average 1 min for a 208×256×256 voxel image. If lookup tables for Dₜ and D_{b} are precomputed and saved, then subsequent executions of the model (e.g. with different values for λ and s, as appropriate for a given tumour) take less than 10 seconds, as long as the tumour and brain segmentations remain unchanged.

### Prior and Data Combination

After registering and deforming the orientation atlas to approximately match the anatomy of the target image, the step to compare the expectation represented in the atlas with the observed dMRI data of the target image is performed (i.e. step S104 in FIG. 1). The orientation atlas is registered from MNI to subject space using affine registration. The tract atlas intentionally conveys a degree of spatial tolerance to account for individual variations in tract location, with the following step acting to refine the estimate according to observed local information in the target image. The objective is to obtain a measure per voxel of how closely the predicted tract orientation distribution overlaps with the observed fibre orientation distribution (FOD), which is modelled from the target dMRI data using (multi-shell multi-tissue) constrained spherical deconvolution (CSD).

This can be achieved by taking the inner product of the two functions, i.e. multiplying them and integrating the product over all spherical angles. The FOD is represented by the modified spherical harmonic (SH) distribution functions as follows, in a similar way to the TOD atlas:

$$F ( θ , ϕ ) = {\sum }_{l = 0}^{{l}_{max}} {\sum }_{m = - l}^{l} {f}_{l , m} {Y}_{l , m} ( θ , ϕ ) = {\sum }_{j} {f}_{j} {Y}_{j} ( θ , ϕ ) ,$$

where Y_{l,m} is the modified SH basis described in Descoteaux et al (Descoteaux, M., Angelino, E., Fitzgibbons, S. & Deriche, R. Apparent diffusion coefficients from high angular resolution diffusion imaging: Estimation and applications. Magnetic Resonance in Medicine 56 (2), 395-410 (2006)). The spherical integral of the product of two spherical harmonic basis functions is ∫₀^{π}∫₀^{2π}Yₗ_{₁}_{,m}_{₁}(θ, ϕ)Yₗ_{₂}_{,m}_{₂}(θ, ϕ)sin(θ)dθdϕ(=δₘ_{₁}_{,m}_{₂}δₗ_{₁}_{,l}_{₂} Therefore, for two functions F(θ, ϕ) and T(θ, ϕ) the integral of their product can be expressed as

$${\int }_{0}^{π} {\int }_{0}^{2 π} F ( θ , ϕ ) T ( θ , ϕ ) sin ( θ ) d θ d ϕ = {\int }_{0}^{π} {\int }_{0}^{2 π} ( {\sum }_{j} {f}_{j} {Y}_{j} ( θ , ϕ ) ) ( {\sum }_{k} {t}_{k} {Y}_{k} ( θ , ϕ ) ) sin ( θ ) d θ d ϕ = {\sum }_{k , j} {f}_{j} {t}_{k} {δ}_{j k} . \qquad ( 7 )$$

Thus, for two distributions each represented by a vector containing their spherical harmonic coefficients, the integrated product can be obtained by calculating the inner product of the two coefficient vectors. That is, the atlas' direction prior consists of a full spherical distribution, instead of a single principal direction per voxel, as for other methods.

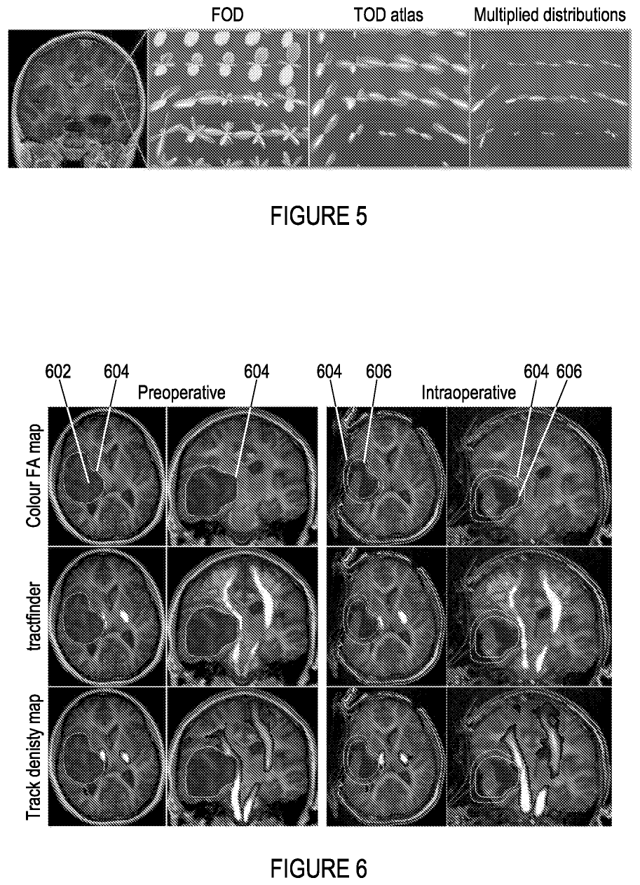

FIG. 5 shows how a modified image of the brain of the subject showing a location and orientation of the specific brain tract is generated. The Figure illustrates atlas and FOD combination, with a close-up of a crossing region between the corticospinal tract (CST) and association fibres of a separate tract. The crossing fibres are visible a green FOD lobes, while branching CST fibres are represented by purple and red lobes. Only directions corresponding to CST fibres are present in the TOD atlas. The multiplication of the two distributions results in suppression of non-CST signal. Integrating the multiplied distributions (inner product) gives the final scalar map (not shown).

Application to Intraoperative dMRI

A proposed pipeline for intraoperative tract segmentation may be as follows, assuming that at least a preoperative structural MRI scan (e.g. TI weighted) is available.

### Preoperative Steps:

1. Register preoperative space to MNI template and transform tract atlas to preoperative space;
2. Segment tumour and brain volume in preoperative scan;
3. Compute tumour deformation field from brain and tumour volumes; and
4. Deform tract atlas

### Intraoperative Steps:

1. Register pre- and intraoperative structural scans and transform intraoperative dMRI scan to preoperative space (2-5 min);
2. Preprocess dMRI data (bias field correction, noise and artefact reduction) (3 min);
3. Construct fibre orientation distribution (FOD) image using CSD (2-3 minutes);
4. Recompute tumour deformation field to account for brain shift (optional) (1 min);
5. Deform tract atlas with updated deformation field (if applicable) (1 min); and
6. Compute inner product tract map (<1 min).

The methodology described above was initially developed and tested in preoperative tumour images. However, the target application is in intraoperative imaging. The main difference therein is the need to account for brain shift, which is unpredictable: differing effects stem from drainage of fluid, pressure changes, tumour debulking and gravitational sag. Nevertheless, the aim is to achieve intraoperative tract segmentation while avoiding the need to perform additional tumour and/or resection cavity segmentation intraoperatively.

As the atlas is designed to be spatially inclusive, with the inner product acting to correct small spatial inaccuracies, it is possible in some cases where brain shift is minimal to reuse the preoperative tumour deformation field. In cases of significant tumour debulking, the deformation field can be recomputed from the preoperative tumour segmentation by adjusting the value of s to simulate a reduction in tumour volume.

This scenario is demonstrated in FIG. 6. FIG. 6 shows use of the present imaging technique to generate an image of brain fibre tracts intraoperatively by adjusting a deformation model that has been obtained preoperatively. In this case, the brain comprises a large temporal epidermoid cyst 602. The images on the left show pre-operative images of the brain, and the images on the right shown intraoperative images, where the surgery involves resection of the cyst. It can be seen by comparing the left and right images that, due to the surgery, there is significant reduction in cyst volume and the adjacent corticospinal tract has shifted accordingly. However, by reusing the preoperative lesion segmentation and setting s=0.8, the resulting deformation field is able to capture the rough location of the shifted tract. In FIG. 6, the boundary labelled 604 is the tumour segmentation, while the boundary labelled 606 is the effective tumour boundary used for intraoperative segmentation (using s=0.8). By only adjusting the value of s and reusing preoperatively computed values of Dₜ and D_{b}, the present techniques advantageously avoid time- and resource-intensive intraoperative lesion segmentation, brain shift modelling or non-linear registration.

FIG. 8A is a flowchart of example steps for generating a tract-specific atlas for imaging brain fibre tracts of a subject. The method shown in the flowchart comprises: obtaining a plurality of images of structurally normal brains of multiple subjects (step S700); extracting, from each image, spatial location and orientation information of at least one brain fibre tract in the brain (step S702); and generating, using the extracted spatial location and orientation information, an atlas comprising a plurality of voxels indicating an expected location and a distribution of expected orientations of at least one specific brain fibre tract (step S704).

Step S704 of generating an atlas may comprise: determining, using the extracted spatial location information from the plurality of images, a likelihood of a specific brain fibre tract being located in a particular voxel.

Step S704 of generating an atlas may comprise: determining, using the extracted orientation information from the plurality of images, a distribution of orientations of a specific brain fibre tract in a particular voxel.

When two or more brain fibre tracts are likely to be located in a particular voxel, step S704 of generating an atlas may comprise: including, in the particular voxel of the atlas, the distribution of expected orientations of each of the two or more brain fibre tracts. In this way, a multi-tract atlas may be generated, which includes location and orientation information for multiple specific tracts individually. Such a multi-tract atlas may be generated by concatenating separate atlases for each specific tract.

Step S700 of obtaining a plurality of images of brains may comprise obtaining images acquired from a high angular resolution diffusion imaging, HARDI, process.

FIG. 8B is a flowchart of example steps for generating a pre-deformed atlas for imaging brain fibre tracts of a subject. The method shown in the flowchart comprises obtaining information about a subject having a brain tumour (step S800). Step S800 may comprise obtaining information on a location of the brain tumour. For example, the information on a location of the brain tumour may be acquired from an image of the brain of the subject. The image of the brain may be a dMRI image, or an image obtained from a structural scan (e.g. a T1 or T2 weighted MRI).

The method of FIG. 8B comprises obtaining an atlas of a specific brain fibre tract of interest, the atlas indicating an expected location and an expected orientation of the specific brain fibre tract in a structurally normal brain (step S802). The techniques to generate such an atlas are described above.

The method of FIG. 8B comprises transforming the atlas, using the obtained information and a tumour model that defines how brain fibre tracts are displaced by tumours, to generate a pre-deformed atlas (step S804). The tumour model may be a radial tumour expansion model, as described above. The tumour model may model how fibre tracts are displaced by infiltrating and/or non-infiltrating tumours.

The atlas may comprise a plurality of voxels. Step S804 may comprise: defining, for each voxel, a distance to a centre of mass of a tumour; and applying, to each voxel, an exponentially decaying function which defines an amount by which each voxel is displaced as being dependent on the distance from the voxel to a centre of mass of a tumour, a distance from the centre of mass to a surface of the brain, and a distance from the centre of mass to a surface of the tumour.

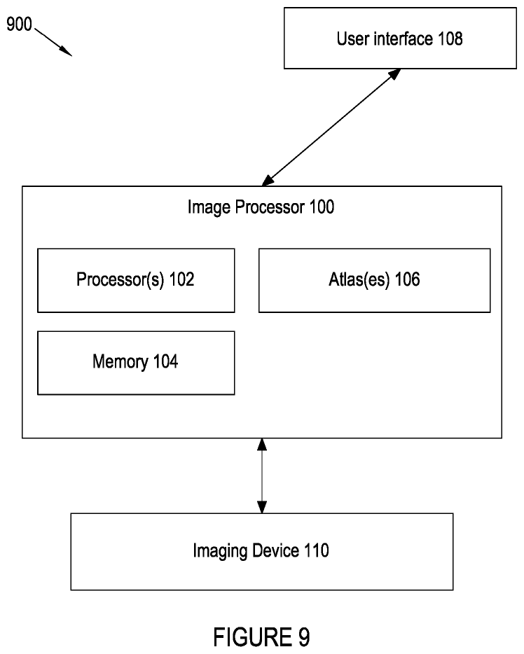

FIG. 9 is a diagram of an image processing system 900 for image processing. The system comprises an image capture device 110 and is configured to capture an image. The image capture device 110 may be a dMRI device.

The system comprises an image processor 100 which is configured to receive an image from the image capture device 110 and carry out the imaging method described herein (e.g. as described with respect to FIG. 1). The image processor 100 comprises at least one processor 102. The at least one processor 102 may comprise one or more of: a microprocessor, a microcontroller, and an integrated circuit. The image processor 100 comprises memory 104 coupled to the at least one processor 102. The memory 104 may comprise volatile memory, such as random access memory (RAM), for use as temporary memory, and/or non-volatile memory such as Flash, read only memory (ROM), or electrically erasable programmable ROM (EEPROM), for storing data, programs, or instructions, for example. The image processor 100 comprises at least one atlas 106. The at least one atlas 106 may represent structurally normal brains and/or represent a brain with a tumour.

The system comprises a user interface 108 which is configured to display an output result generated by the image processor 100. The user interface 108 may be part of the image processor 100. The user interface 108 may be a display device, for example.

### Results and Discussion

Due to the lack of ground truth information for white matter tract segmentation in in vivo dMRI images, especially in neurosurgical cases, a quantitative validation of this method is not currently possible. However, comparison with the current clinical standard, streamline tractography, illustrates the effectiveness of tractfinder. Targeted probabilistic streamline tractography reconstructions were produced by an experienced operator utilising ROI placement strategies routinely utilised in clinical practice at the inventors' institution. The clinical reliability and biological accuracy of tractography is difficult to determine in vivo. While some consider the “gold standard” to be validation against intraoperative direct electrical stimulation and post-surgical outcomes, which is unavailable for the presented data, even this can provide only incomplete information on spatial accuracy.

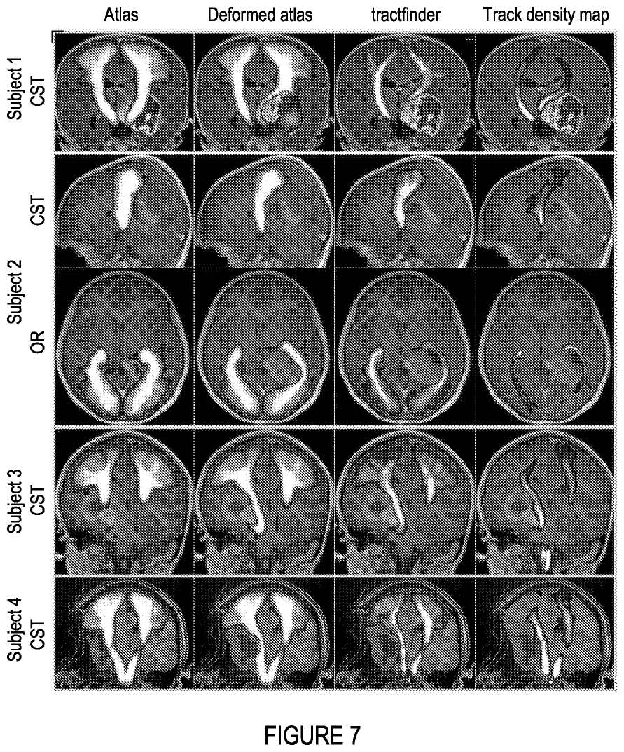

FIG. 7 shows results for four different example subjects (three paediatric and one adult) with space occupying tumours. In FIG. 7, the first column shows a linearly registered tract atlas having a spatial probability component only. The second column shows the atlas after tumour deformation. The third column shows the final tract map, and the fourth column shows track density images from streamline tractography, where intensity corresponds to streamline count per (2.5 mm)³ voxel (thresholded at 10 streamlines). In each subject j, the value of λ varies spatially and was set automatically to min{λⱼ, λₘₐₓ} according to Equation (4), with λⱼ=8 everywhere except for subject 2, optic radiation, where λⱼ=2. In subjects 1-3, s=1, while in subject 4, s=0:8 (see also FIG. 6). In FIG. 7, CST means corticospinal tract, and OR means optic radiation.

These initial results serve to demonstrate the feasibility of the proposed method (demonstrated in the corticospinal tract in all four subjects and additionally the optic radiation in subject 2) in a range of complex clinical cases.

The tumour deformation model successfully captures large-scale tract displacements in seconds, where much longer timescales (several minutes to hours) are typical for more complex tumour growth modelling algorithms and non-linear registration. The short computational time further makes it trivial to recompute the deformation with small adjustments if necessary. The model presents a simplified prediction of tumour deformation: No distinctions are made between the highly deformable ventricles and stiffer brain tissues, and the tumour is “grown” isotropically from a single point outward with no regard for the surrounding topology (except for the brain boundary) or peri-tumoural tissue effects. Nevertheless, the objective of the deformation step, which is to bring the tract orientation atlas into approximate alignment with the actual target tract, is achieved despite these simplifications. Improvement is needed in cases involving infiltrative tumours, where tracts are not entirely displaced and tumour cells mix with surrounding functional structures, as the current model only supports single tumours with defined boundaries. Modelling this scenario will require a modified deformation model, using a different expression for k(P).

The inner product between the orientation atlas and target FOD image provides an intuitive map of tract location and is computationally straightforward (FIGS. 5 and 7). Successful results have been obtained in clinical quality, single-shelled diffusion MRI datasets. However, there remains the need to more thoroughly explore the effects of different acquisition protocols, including fewer diffusion encoding directions and lower b-values, on segmentation quality. So far, there has been limited validation of applying tractfinder to intraoperative cases, and this will be the subject of future research. One example of such a case is shown in FIG. 6. Using a lesion shrinkage factor of s=0.8 is successful at creating a deformation field that corresponds with the intraoperative anatomy, and the resulting map of the CST captures the tract's course at the edge of the lesion and resection cavity.

If implemented clinically, intraoperative processing steps would be limited to minimal preprocessing including de-noising and bias field correction, registration to preoperative data, followed by FOD modelling, adjustments to tumour deformation modelling if necessary and inner product computation. Other preprocessing steps which are routine in preoperative and research imaging contexts, such as correction for eddy current and geometric distortion artefacts, have been omitted due to long processing times making them impractical for intraoperative use. Future research should investigate the implications of omitting these corrections and possible more lightweight implementations. Total processing time for the above steps should not exceed 15 minutes, and could be completed in parallel with the non-diffusion iMRI acquisition protocol (if the site-specific setup allows parallel acquisition and data processing), which can take up to 50 minutes. Operator input and time is currently required preoperatively for manual tumour segmentation, although this step could feasibly be automated given the extensive interest and research into automatic brain tumour segmentation. Additionally, all intraoperative processing steps can be completed automatically using default values. The process is no longer fully automatic if manual adjustments to λ and s are necessary, however this nevertheless amounts to far less operator input than advanced streamline tractography as described in the introduction. A final critical practical aspect of intraoperative implementation will be the integration of segmentation results with neuronavigation tools for display during surgery, including the appropriate data conversions. Tools to enable such interfacing have been developed by others and could likely form part of the full clinical tractfinder implementation.

In conclusion, a white matter mapping method is presented that is shown to produce plausible tract reconstructions in cases with space occupying lesions, using an atlas in conjunction with tumour deformation modelling. Producing results requiring minimal user input and on intraoperatively feasible timescales, the method thus has the potential to bring effective white matter mapping into the intraoperative domain.

The final result is this voxel-wise inner product of the registered atlas and subject FOD images. The resulting image is a pseudo-probability map of tract location, in arbitrary and dimensionless units. Typical values range from [0-0.5], with 0.05 empirically determined to be a suitable threshold for converting to binary segmentation.

### Validation

While a ground truth for white matter tract segmentation is not obtainable in vivo, the present techniques are compared with two other widely adopted methods for a quantitative estimation of reliability and accuracy. Results are presented for three different datasets: Two large healthy datasets and one smaller dataset of clinical neurosurgical acquisitions, together covering a range of acquisition protocols and scanner specifications. In segmentation tasks, it is common to present a single numeric score of similarity with a ground truth by way of establishing accuracy. However, in the absence of a ground truth for this particular task, the aim is to present as rounded a picture as possible of the differences and characteristic features of each method through a range of different volumetric distance-based similarity metrics. The purpose of this validation is therefore not to determine which method is best, as indeed cannot be determined without a reliable reference point, but to highlight the ways in which they are similar, and their characteristic tendencies.

Data: Three different datasets were used to compare the proposed method against alternative tract segmentation methods. Each dataset and any dataset-specific preprocessing is described below. In addition, for all subjects brain masking was performed (J-Donald Tournier et al. 2019) linear registration(Jenkinson and Smith 2001; Jenkinson et al. 2002) (FSL v.6.0 Linear Image Registration Tool) between subject space and MNI152(Fonov et al. 2011) space and two versions of constrained spherical deconvolution (CSD): single-shell, single-tissue (SSST) CSD (“original flavour”)(J. Donald Tournier, Calamante, and Connelly 2007; J-Donald Tournier et al. 2019) and multi-shell, multi-tissue CSD(Jeurissen et al. 2014) restricted to white matter and grey matter tissue compartments. In both cases, response functions were obtained using the Dhollander unsupervised 3-tissue response function estimation algorithm.(Dhollander, Raffelt, and Connelly 2016; Dhollander et al. 2019) All CSD processing was conducted using the MRtrix3 image processing software package v3.0.2-3.0.3 (https://www.mrtrix.org/).(J-Donald Tournier et al. 2019) Processing and analysis pipelines for the two openly available datasets are available at https://github.com/fionaEyoung/pipelines

HCP: 49 scans were accessed from the WU-Minn HCP Young Adult S1200 data release (https://www.humanconnectome.org/study/hcp-young-adult/document/1200-subjects-data-release) (Van Essen et al. 2013). These images have been preprocessed as documented in Glasser et al. (2013)(Glasser et al. 2013) (Glasser et al. 2013). The scans were downsampled to 2.5 mm isotropic voxels and a subset of 60 directions were extracted at b=1000 mm/s².

TractoInferno: The recently released TractoInferno database (v.1.1, available at https://openneuro.org/datasets/ds003900/versions/1.1.1),(Poulin et al. 2022) created for the training of machine learning tractography approaches, contains diffusion and T1-weighted MRI scans for 284 subjects pooled from several studies, accompanied by reference streamline tractography reconstructions. Of the 284 subjects included in the full TractoInferno database, 144 subjects were selected with tractography of the CST, OR and AF for the study. Nine subjects were excluded from the final analysis due to inadequate non-linear registration performance resulting in failed in-house tractography, leaving a final 135 subjects. Diffusion acquisition parameters and preprocessing steps are described in Poulin et al. (2022)(Poulin et al. 2022) (Poulin et al. 2022), and additionally all data was resampled to 2.3 mm isotropic voxels, the lowest resolution present in the dataset and one in line with clinical acquisitions.

Clinical: Tract segmentation comparisons are presented for 15 individual scans from eight different subjects from two different institutions. They include four adult glioma subjects acquired in 2009 at the National Hospital for Neurology and Neurosurgery, London (NHNN) (cases 4 and 5 from (Mancini2022?)(Mancini2022?) (Mancini2022?), others unpublished data), three paediatric subjects from Great Ormond Street Hospital, London (GOSH) (each with one preoperative and one intraoperative scan), and a mock “intraoperative” scan on a healthy adult volunteer acquired with the GOSH intraoperative DTI protocol and using simulated intraoperative setup (flex-coils wrapped around the head instead of a head coil, head significantly displaced from scanner isocenter etc). For acquisition details see Table [tab:datasets]. All clinical scans involved non-deforming tumours, in the sense that any lesions did not appreciably displace white matter structures from their typical positions.

This study and the use of GOSH clinical data was approved by UCL REC (ID2780/003) and the UCL Institute of Child Health/GOSH joint R&D office (reference 19NI12). Use of NHNN data was approved under retrospective research ethics by the NHNN (University College London Hospitals NHS Foundation Trust) and UCL Institute of Neurology Joint Research Ethics Committee (REC 18/NW/0395, IRAS No: 213920). In addition, the acquisition and use of some NHNN MRI data was also approved by the NHNN (University College London Hospitals NHS Foundation Trust) and UCL Institute of Neurology Joint Research Ethics Committee (REC 12/LO/1977). All clinical data was acquired within the course of routine clinical care, and as no identifying information of any subject is present, there is no need for informed consent. To protect patient confidentiality, clinical data will not be made openly available.

Each dMRI scan was minimally preprocessed with Marchenko-Pastur principal component analysis denoising(Veraart et al. 2016; Cordero-Grande et al. 2019) Gibbs-ringing correction(Kellner et al. 2016) and bias field correction,(Zhang, Brady, and Smith 2001; S. M. Smith et al. 2004) as implemented in MRtrix3 (J-Donald Tournier et al. 2019). Preoperative scans additionally had eddy current and motion distortion correction(Andersson and Sotiropoulos 2016; S. M. Smith et al. 2004) (MRtrix3 v3.0.3 and FMRIB Software Library (FSL, https://fsl.fmrib.ox.ac.uk) v6.0) applied, while this step was omitted for intraoperative scans to maintain a clinically realistic timeline. No EPI distortion correction was performed, as it is frequently omitted from clinical pipelines due to lack of requisite reverse phase encoding or field map information and long processing times.(Yang et al. 2022)

### Compared Methods

Three alternative segmentation approaches were considered and compared each with the proposed method: Probabilistic streamline tractography, representing the current standard, the deep learning direct segmentation technique TractSeg, and a “naive” atlas registration.

Streamline tractography: Targeted probabilistic streamline tractography (iFOD2 algorithm(J.-D. Tournier 2010) was run, from MRtrix3(J-Donald Tournier et al. 2019) v3.0.3) in each scan using an in-house ROI strategy (see 6.1 for ROI details), with tractography input FODs derived from multi-shell, multi-tissue CSD (Jeurissen et al. 2014) with white matter and grey matter tissue compartments. In the clinical dataset, ROIs were placed manually for each subject. For 193 HCP and TractoInferno subjects, manual ROI placement was infeasible. Instead the same ROIs were drawn in MN1152 space aided by the FSL HCP-1065 DTI template(“Hcp1065 Standard-Space DTI Templates” n.d.) and transformed to subject space using non-linear registration (HCP data includes MNI transformation warps, while warps were created for the TractoInferno data using the ANTs registration package v2.4.2 (http://stnava.github.io/ANTs/).(Tustison and Avants 2013; Avants et al. 2011)). This in-house tractography is subsequently abbreviated to “TG”, while the reference TractoInferno bundles are referred to as “TGR”.

TractSeg: TractSeg (Wasserthal, Neher, and Maier-Hein 2018) is a deep learning tract segmentation model which produces volumetric segmentations for 72 tracts directly from fibre orientation distribution peak directions (TractSeg v2.3-2.6, available at https://github.com/MIC-DKFZ/TractSeg). There are two models available: one (“DKFZ”) trained on modified streamline reconstructions using TractQuerier (Wassermann et al. 2016) as described in Wasserthal, Neher, and Maier-Hein (2018)(Wasserthal, Neher, and Maier-Hein 2018) (Wasserthal, Neher, and Maier-Hein 2018), and a second (“XTRACT”) trained on streamline density maps output by FSL's XTRACT application. (Warrington et al. 2020) Comparisons with both versions were made, as they feature significant differences in anatomical tract definition. Input peaks were derived from the SSST CSD FODs.

Atlas registration: As well as the full tractfinder method, the results were compared with a “naive” tract atlas approach, taking the density component (first SH coefficient) of the linearly registered tract atlases. This amounts to a segmentation based on prior expectation only, without taking into account the diffusion data.

Comparison metrics: Each technique was compared against the others, rather than designating any single technique as “ground truth”. Several comparison metrics were computed, to capture different kinds of agreement between segmentations. The Dice-Soerensen similarity coefficient (DSC) is a popular, symmetric measure of segmentation similarity given by

$$D S C = \frac{2 \vert A ⋂ B \vert}{\vert A \vert + \vert B \vert}$$

for two binary voxel sets A and B. Since DSC is a measure for binary segmentations, it requires the thresholding of continuous-valued maps such as track density maps and the pseudo-probability maps produced by tractfinder. Firstly, the conversion from continuous-valued to binary representation introduces a high degree of ambiguity over the appropriate choice of threshold value. While the simplest approach may be to include all voxels with value >0 in the segmentation, this makes little sense in practice. In the case of tractography, a small number of rogue false positive streamlines can massively increase the extent of the binary segmentation, and in the case of TractSeg, very few voxels actually are assigned a probability of 0. The following thresholds were used throughout, wherever binary segmentations are concerned:

| Method | Threshold value |
|---|---|
| Tractfinder | 0.05 |
| Tractography (streamline density) | 10 |
| Reference tractography (TractoInferno | 0 |
| only) | |
| Atlas | 0.1 |
| TractSeq | 0.5 |

Secondly, the binary nature of DSC discounts the additional confidence information present in density and probability maps. In addition to the binary DSC measure, therefore, a generalisation of the DSC (gDSC) for continuous valued segmentations was considered.

$$g D S C = \frac{2 {\sum }_{i} \sqrt{{a}_{i} {b}_{i}}}{{\sum }_{i} {a}_{i} + {\sum }_{i} {b}_{i}} = \frac{2 {\sum }_{i} \sqrt{{a}_{i} {b}_{i}}}{{‖ a ‖}_{1} + {‖ b ‖}_{1}}$$

The density correlation metric provides an alternative measure of agreement between two continuous valued segmentations with different scales: it is simply the Pearson correlation coefficient between the two sets of voxel values.

In addition to the volumetric overlap and density metrics DSC, gDSC and density correlation, the volumetric bundle adjacency was measured as defined in: “Tractography dissection variability: What happens when 42 groups dissect 14 white matter bundles on the same dataset?” by Schilling et al. published in NeuroImage 243, 118502. doi:10 0.1016/j.neuroimage.2021.118502 in 2021, which is referred to as the bundle distance BD. It is computed by taking the mean of minimum distances from every non-overlapping voxel, in each segmentation, to the closest voxel in the other segmentation. Finally, to give a sense of whether the boundary of one segmentation is within or outside that of a second segmentation, the signed bundle distance BDₛ was also measured. This metric is asymmetric, with BDₛ(A,B)=−BDₛ(B,A). Thus BD and BDₛ are defined as

$$B D ( A , B ) = \frac{{\sum }_{i ∈ A ∖ B} {d}_{i} ( B ) + {\sum }_{i ∈ B ∖ A} {d}_{i} ( A )}{\vert A Δ B \vert}$$

$$B {D}_{s} ( A , B ) = \frac{{\sum }_{i ∈ A ∖ B} - {d}_{i} ( B ) + {\sum }_{i ∈ B ∖ A} {d}_{i} ( A )}{\vert A Δ B \vert}$$

where |⋅| denotes set cardinality and dᵢ(X) is the Euclidean distance transform (defined relative to the foreground of segmentation X, i.e. dᵢ(X)=0 when i∈X and dᵢ(X)=|\vec{ij}| when i∉X and where j∈X is the voxel in X closest to voxel i [symbol] of segmentation X at voxel i.

### Results

Atlas: Despite careful manual filtering, it isn't possible to completely remove all spurious streamlines from the bundles used to construct the atlases. In particular, SLF and tapetum of the corpus callosum contamination in the optic radiation bundles proved difficult to eliminate, despite removing up to 20% of streamlines.

Processing times: Atlas transformation and inner product computation time per subject for all three tracts and both hemispheres was 18±5s, plus 1-2 minutes for MSMT-CSD and 20 seconds for MNI registration. For TractSeg (DKFZ or XTRACT), mean processing time (for all tracts, 72 for DKFZ and 23 for XTRACT, both hemispheres) was 4:00±1:00 min, plus 15-20s for SSST-CSD.

For manual streamline tractography, processing time was not explicitly measured, due to the high variability that comes with manual ROI drawing (between 10-25 minutes for all tracts in a single subject, although this varies significantly between operators). HCP and TractoInferno tractography was run on a high performance computing cluster, taking approximately 10s per tract (single hemisphere), using 36 CPU cores, and additionally up to 2 minutes for non-linear ROI registration. However, since the time taken depends greatly on several factors, including number of streamlines to select and streamline acceptance rate (often low in pathological brains due to oedema, deformation etc.), a detailed time analysis for manual tractography is not provided here.

### Qualitative Results

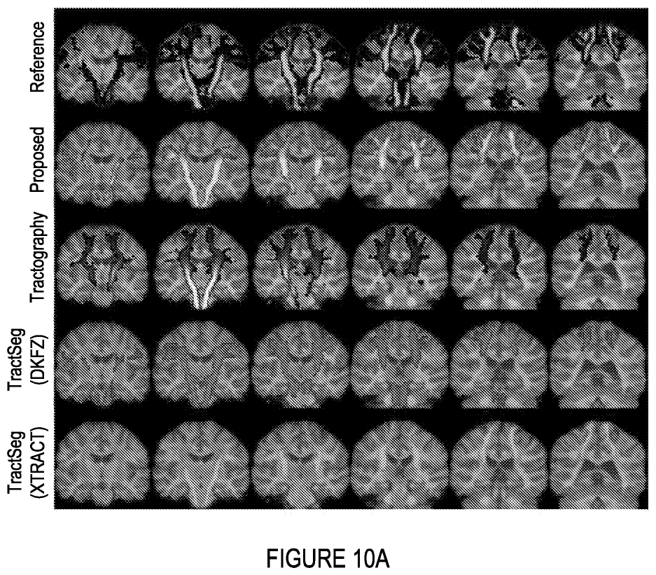

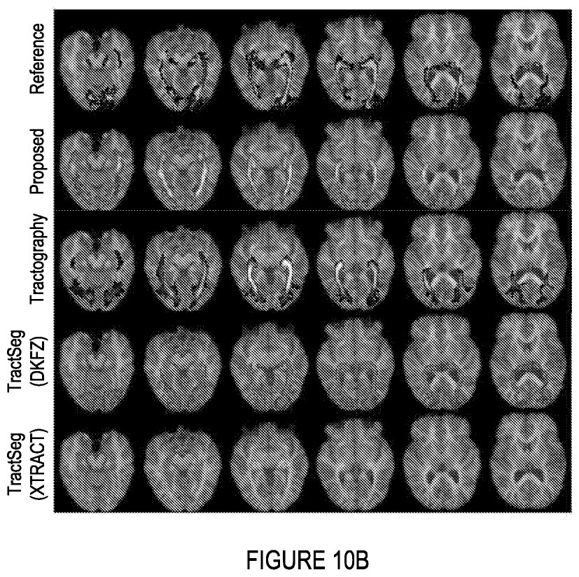

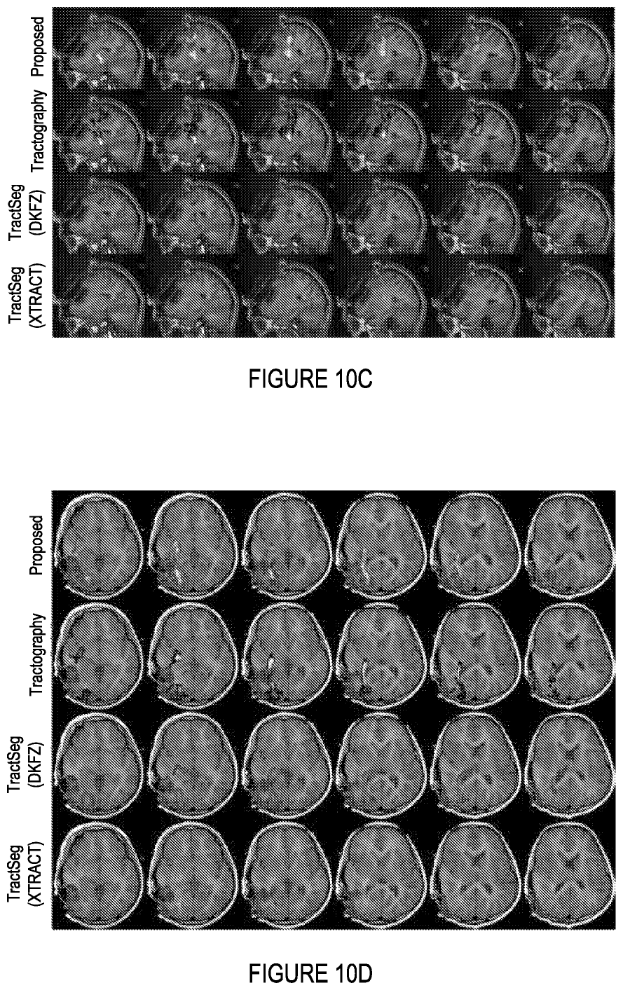

FIGS. 10a to 10d show qualitative results obtained using the techniques of FIG. 1. The raw tract maps typically have values ranging from 0 to 0.5 (in arbitrary units, derived from the magnitudes of FOD and atlas distribution functions). Due to the combined effects of ODF amplitude and orientation information, a low tract map value can have several causes: a) the FOD amplitude is low, indicating low evidence for white matter tissue in the voxel in question; b) the atlas amplitude is low, indicating low prior likelihood of the tract being present in that location; c) the peak orientations between the FOD and atlas are poorly aligned.

Thus, combining information from the atlas and data-derived FODs improves the tract map estimation over the “raw” registered atlas in both the spatial and orientational domain. For example, the TOD atlases have poor definition of gyri and sulci, due to the effect of averaging over many subjects and linear registration. The reduced overall FOD amplitude in grey matter corrects this non-specificity. And in regions where different white matter structures lie in close proximity, where the atlas can erroneously predict the likely presence of the tract, and FOD amplitude is high, the lack of orientational agreement discounts the presence of the tract of interest in that location.

Quantitative results in healthy data: Volumetric and agreement metrics indicate consistent, if not always high, levels of agreement between tractfinder and compared techniques, TractSeg and tractography. Visual assessment reveals that differences in the spatial extent of the segmented tracts accounts for a large part of the discrepancy between methods. This is most apparent in the arcuate fasciculus, where anatomical definitions differ widely. For example, TractSeg (DKFZ) includes extensive coverage of the frontal and temporal lobe in its AF segmentations, including parts of the primary motor cortex. Conversely in the corticospinal tract, which has a relatively well agreed-upon domain, segmentation results have much higher volumetric agreement between methods.

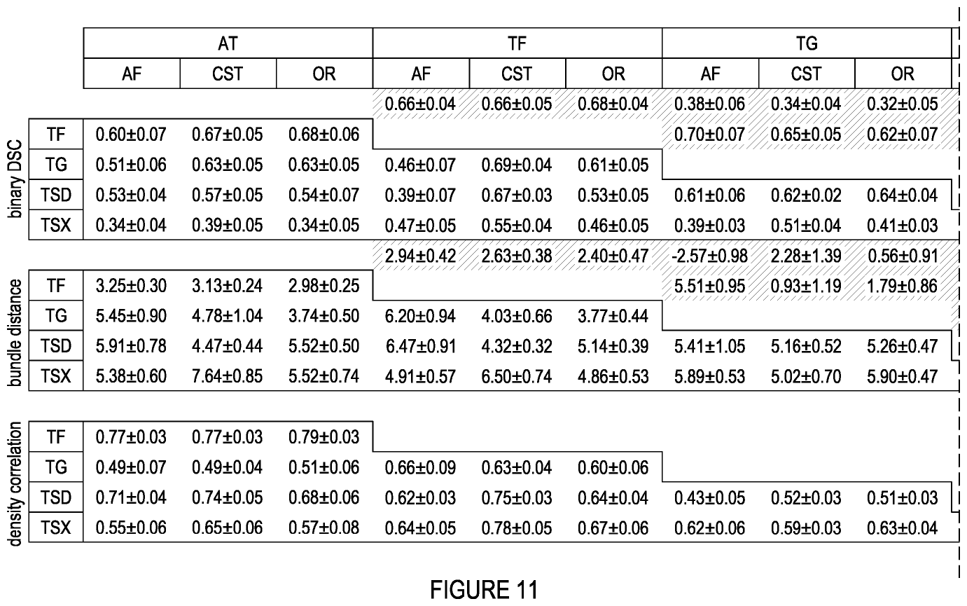

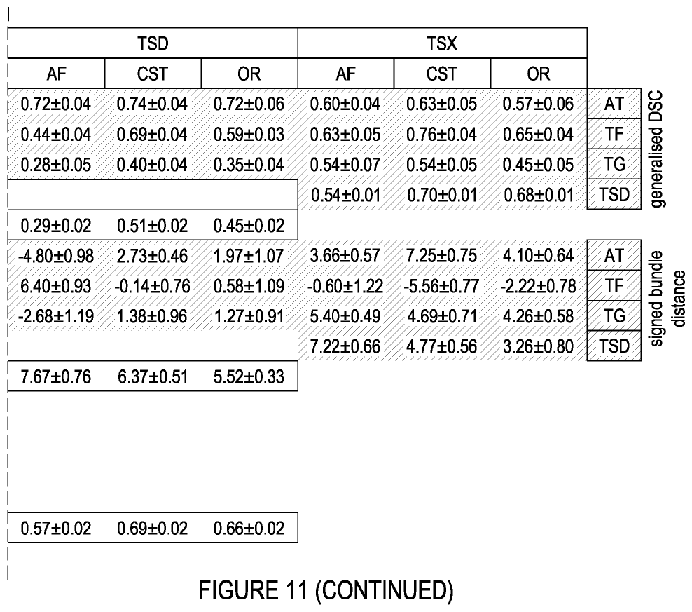

FIG. 11 is a table showing all pairwise comparisons for the HCP dataset. Bundle distances are given in mm, with all other metrics being dimensionless. The abbreviations used in the table (and throughout the present application) are: AF=arcuate fasciculus; CST=corticospinal tract; OR=optic radiation; TF=tractfinder (proposed); TGR=reference tractography (TractoInferno streamlines); TG=in-house tractography; TSD=TractSeg (DKFZ); TSX=TractSeg (XTRACT); and AT=atlas.

The signed bundle distance gives an indication of the nature of disagreement between two techniques where other metrics show little difference. For example, in the HCP dataset and for the arcuate fasciculus, mean bundle distance between the naive atlas and tractography was 5.45 mm and mean bundle distance between TractSeg (DKFZ) and tractography was very similar at 5.41 mm. However, the signed bundle distances for those same two comparisons were +2.57 mm and −2.68 mm respectively. This indicates that, while if only considering the bundle distance metric, both TractSeg and the atlas appear to agree to a similar degree with tractography, TractSeg actually systematically over-segments the AF (relative to tractography), while the naive atlas segmentation tends towards under-segmentation.

Density correlation and gDSC help illustrate the cases where the choice of threshold may have a disproportionate influence on subsequent binary comparisons. For example, in the HCP dataset and for the corticospinal tract, mean binary DSC was 0.69 between tractfinder and tractography and 0.51 between TractSeg (XTRACT) and tractography (a difference of 0.18). For the same two comparisons, the density correlations differed only by 0.04 (0.63 and 0.59) respectively, indicating strong agreement between areas of high confidence (“density”).

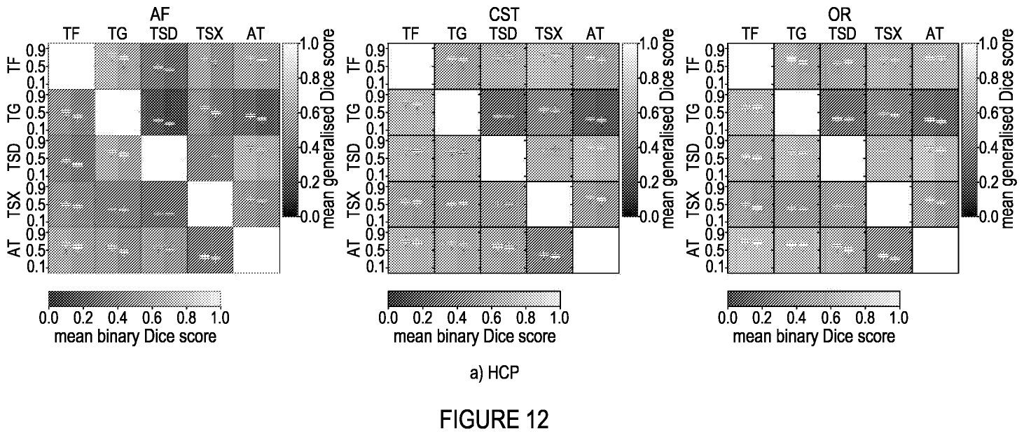

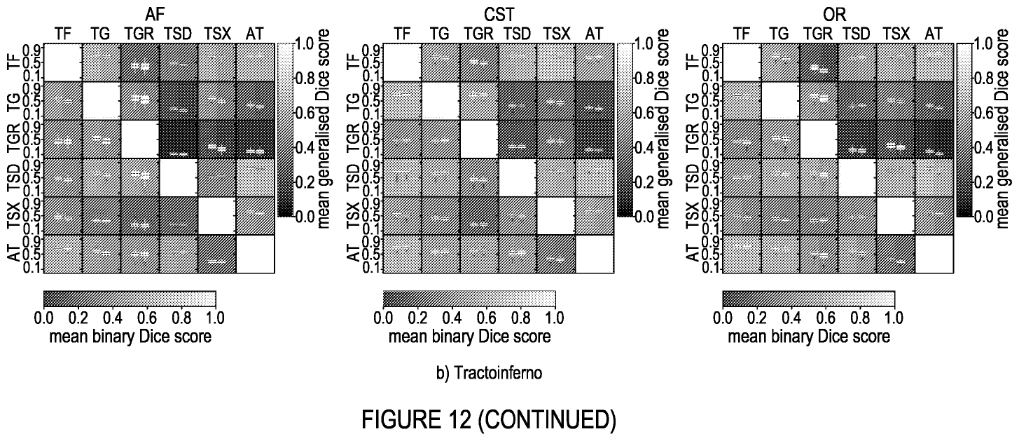

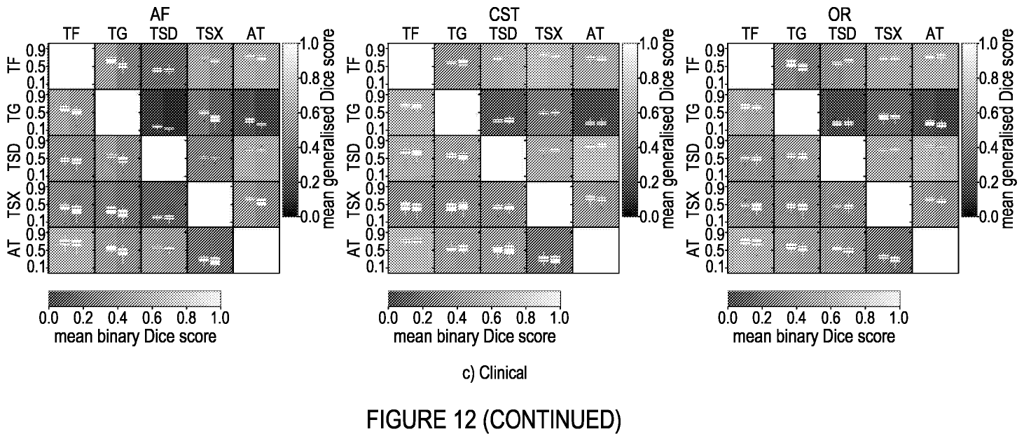

HCP data: FIGS. 12a to 12c give an indication of how the five segmentations stack up against each other, across all HCP dataset subjects. There is considerable variance between tracts, however some observations remain consistent.

Comparisons with tractography exhibit very low gDSC values. Binary DSCs are low across the board for the arcuate fasciculus, owing to the dramatically different spatial extents of the segmentations. For the corticospinal tracts, tractfinder agrees relatively strongly with each of the other methods, both using binary and generalised comparisons. Agreement is similarly high in the optic radiations, with slightly lower DSCs compared to the two TractSeg methods, which tend to include more thalamus and a lesser extent of Meyer's loop.

Tractfinder segmentations are highly consistent, with comparison metrics with alternative methods varying by little across subjects, as shown in FIG. 11.

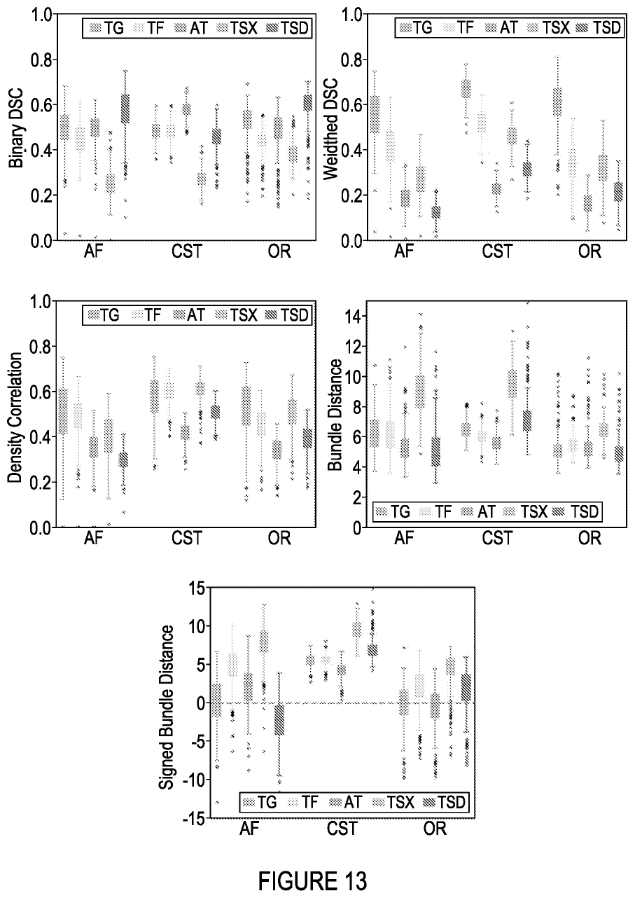

TractoInferno: FIG. 13 compares each studied method against the reference streamline bundles in the TractoInferno dataset. Noticeably, the differences in scores within a single method, between different tracts, are in places greater than the differences between methods within a tract. For example, the binary DSC scores for the CST are similar for tractfinder and TractSeg (DKFZ) (0.48 and 0.45 on average respectively), however the binary DSCs of TractSeg (DKFZ) are markedly different between the CST and OR (0.45 and 0.59 on average respectively). These differences highlight the difficulty in assessing the “accuracy” of white matter segmentation methods given limited consensus on the precise anatomical definitions of different pathways. DSC, gDSC and density correlation values for tractfinder were on par with TractSeg (XTRACT) in all three tracts, with the exception of density correlation in AF, while gDSC and density correlation were higher than TractSeg (DKFZ) in all tracts. Binary DSC scores were highest for TractSeg (DKFZ) in the CST and AF, and equal between tractography, tractfinder and TractSeg (DKFZ) for the optic radiation. The results in FIG. 13 are consistent with the comparisons between TractSeg and RecoBundles published in: “TractSeg—Fast and accurate white matter tract segmentation” by Wasserthal et al. published in NeuroImage 183, 239{253.doi:10.1016/j.neuroimage.2018.07.070. pmid: 30086412 in 2018. There, a mean DSC of between 0.58 and 0.67 across all tracts was reported. The measured DSCs between TractSeg (DKFZ) and reference tractography (which is based on RecoBundlesX(Garyfallidis et al. 2018)) range between 0.45 and 0.59 across the three tracts studied.

From FIG. 13, it is apparent that comparisons with TractoInferno reference streamlines yield a large number of outliers and low scores. Further investigation into these outliers revealed numerous subjects with incomplete or highly asymmetric bundles. For example, in several cases, optic radiation streamlines only reach the superior portion of the occipital lobe. In others, the right arcuate fasciculus is significantly smaller than the left. There is lower variability in the pairwise comparisons between the other four methods: the results for tractfinder and TractSeg remain in more consistent agreement with each other across the TractoInferno dataset, as shown in FIG. 12b.

Results in clinical data: For the present analysis clinical scans with non-deforming lesions are included, meaning the orientation atlas could be registered to the target image using only affine registration without the need for tumour deformation modelling.

In the clinical dataset, mean values of agreement with other segmentation methods were consistent with those in the ideal, healthy datasets, while variability between subjects was higher, as shown in FIG. 12c. Again, comparisons between the segmentation methods vary significantly between tracts. The size of this dataset is far smaller than the other two, but even so the results are consistent with those seen for the larger, healthy subject datasets.

Two example clinical subjects, one adult and one paediatric, are displayed in FIGS. 10c and 10d. In FIG. 10c, a sagittal view displays the interaction between the surgical resection cavity and the CST. Here the proposed method maps the CST in relatively close proximity to the resection site, where the TractSeg segmentations are far more conservative, potentially missing CST locations influenced by oedema or other tumour effects. In FIG. 10d, the extent of Meyer's loop depicted by tractography is similarly included in the proposed segmentation, but absent from the TractSeg results.

When the results for the clinical dataset were split on hospital/age group (paediatric or adult), no appreciable difference in results was observed (data not shown). Equally, no systematic difference was observed between intraoperative and preoperative datasets.

### Discussion

A full methodological description and validation of a novel white matter segmentation technique is presented here, which can produce consistent results with minimal user input. Accuracy, so far as can be measured, is comparable with alternative techniques, with differences in comparison metrics being driven primarily by disagreements in tract definitions, rather than by methodological performance.

The inner product between the orientation atlas and target FOD image provides an intuitive map of tract location and is computationally straightforward. An advantage of tractfinder over a deep learning method is the element of explainability that is provided by the orientation atlas and subsequent combination with the data. The simple mathematical formulation affords an intuitive understanding of why a given voxel is included in a segmentation, and can be visualised along with the subjects FODs for additional clarity.

There are logistical barriers to deployment of tractography in neurosurgical units in the United Kingdom, as well as a lack of knowledge among neurosurgeons of the underlying choices in fibre modelling and tracking algorithms involved, which could lead to flawed interpretations of tractography reconstructions. It is hoped that the use of a predefined tract atlas in combination with readily automated registration and dMRI processing tools will go some way towards reducing that logistical burden, as well as aiding interpretation of the results.

It is important to note that when it comes to white matter segmentation, there is as of yet no one technique to rule them all: Each has individual strengths and weaknesses and are appropriate for different context, depending on constraints on processing time, computer power, operator expertise as well as the context of application (e.g. whether streamlines are required or voxel-wise segmentations). Tractfinder, as well as being generally applicable to healthy datasets, has been developed specifically with a neurosurgical context in mind, and can flexibly accommodate minor tumour distortions and epilepsy pathologies out-of-the-box, and larger distortions with additional adjustment. There is also qualitative evidence that it can produce reconstructions with sensitivity more comparable to that of tractography than TractSeg, in situations of surgical relevance, such as the proximity of the CST to the resection site in FIG. 10C, or the extent of Meyer's Loop, a structure with frequent significance for postoperative visual field deficits, in FIG. 10D.

Comparison with alternative segmentation methods: Comparing methods as varied in approach as direct atlas based segmentation, deep learning inference and streamline tractography makes objective comparison difficult. Qualitatively, the spatial coverage for all three tracts shown is well-matched with streamlines, while TractSeg (DKFZ) generally produces broader segmentations.

Multiple quantitative volumetric similarity metrics are presented to give a rounded picture of the differences in results, as no single measure can helpfully capture all aspects of complex tract segmentations.

Tractfinder is compared with three other segmentation approaches to the reference streamline bundles in the TractoInferno dataset. Among all metrics and tracts, the large range of values indicates either a high degree of variability in the reference streamline bundles, or a low level of robustness in all of the investigated methods. Some inconsistencies in the reference bundles resulted in a large number of outliers in comparisons with all four other methods. These results have been included here as we wanted to take this dataset of reference bundles, which has been published for the purpose of training and validating machine learning tractography algorithms, “at face value”. They demonstrate how even with diligent manual quality control, achieving consistent, reproducible white matter segmentation results in hundreds of subjects using streamline tractography remains extremely difficult.

In FIG. 13 the present approach is compared with three other segmentations with the TractoInferno reference bundles. However, it is important to avoid interpreting these comparisons as measures of “accuracy” for the respective methods. Take, for example, the higher binary DSC (0.58) obtained by a naive atlas registration, without incorporating any subject diffusion information, compared to TractSeg (DKFZ) (0.45) for the CST. Consider also the large differences between the TractSeg (DKFZ) and TractSeg (XTRACT) results, which demonstrate how the underlying tract definitions from which a prior is derived (e.g. an atlas, deep learning model, etc.) is the driving factor in dice score “accuracy”. Numerical metrics can thus highlight tendencies and help determine which methods perform consistently, while visual assessment remains the only way of gauging anatomical accuracy.

Segmentation approaches in two other datasets with no reference bundles were also compared: a set of HCP subjects and a selection of clinical subjects. The problem of defining reference bundles for the validation of tract segmentation still needs to be properly addressed by the diffusion imaging community. It is a two-fold problem: a lack of both a sufficiently accurate and reliable method and of agreed upon anatomical definitions (see below). Some tract segmentation studies define their own reference bundles used for both model training and evaluation. While the in-house tractography results presented for the clinical and HCP datasets represent the current standard process, treating them as reference bundles could have produced biased interpretations, as the same ROI strategy and anatomical interpretation were employed to produce the tract atlases. Instead, only pairwise comparisons between all techniques and qualitative results are presented. It is for the same reasoning that the publicly available bundles for the 105 HCP subjects on which TractSeg (DKFZ) was trained are not included as additional reference bundles for the HCP dataset in the analysis. It is also noted that, without knowledge the subject IDs of the TractSeg training dataset, it is likely that some of the subjects in the HCP dataset have already been “seen” by TractSeg during training.

In the HCP dataset, tractfinder agrees strongly with the other methods, particularly in the corticospinal tracts. TractSeg (XTRACT) stands out as having low binary DSC scores when compared with the other methods. Comparisons with streamline tractography and non-tractography methods generally exhibit very low gDSC values. This is presumed to be due to the extreme density bias common in TDI maps, with values within a relatively small central portion of the tract being orders of magnitude greater than in the periphery.

Data requirements results: the effect of reduced number of diffusion weighting directions and different fibre orientation distribution reconstruction approaches on segmentation stability have previously been investigated for the segmentation methods analysed here. Segmentation results using tractfinder remained consistent within a subject even with decreasing data acquisition quality, from 120 directions and two (nonzero) b-value shells down to as few as 12 directions at b=1000. The ideal acquisition is at least 60 diffusion directions at b=1000, although fully acceptable results can be obtained with just 30 directions, as in the presented intraoperative data.

The current analysis also indicates that the results are not beholden to a specific preprocessing pipeline: in the clinical dataset, differences in preprocessing steps yielded no discernible effects on resulting segmentations.

Limitations: An atlas-based tract segmentation method such as the one proposed here is not suitable for “exploratory” connectivity studies, where tract segmentations and connections are sought without imposing any prior expectations. Instead, this approach is more suited for applications where tract segmentations are unlikely to deviate from expectations, and where rapid and robust segmentation is a priority.

Owing to the intensive process of constructing a new atlas, validation of the present approach was focussed on a few tracts before moving on to constructing further atlases. The availability of atlases for the CST, AF and OR only limits the applicability of the present technique to those three tracts for the time being, whereas many alternative methods can segment tens of different pathways at a time. The construction of atlases for other commonly studied pathways, such as the IFOF and UF, will be the subject of future development.

The current approach of taking the inner product of the two angular distributions produces good results, is computationally straightforward, and has intuitive meaning. However, a potential drawback, depending on the desired information to be provided by tract mapping, is the behaviour in the presence of multiple fibre populations. The presence of crossing fibres will reduce the map amplitude, even if one of the FOD fibre populations aligns well with the atlas, as the presence of the crossing fibre population reduces the overall inner product value. If the objective is to obtain a likelihood score for a particular tract, regardless of whether or not the tract is sharing a voxel with another fibre population, then the current inner product framework, which slightly penalises crossing fibre voxels over single fibre ones, is inadequate.

Tractfinder requires an additional registration step and relies on good alignment of the atlas to subject data, although in this regard affine registration is sufficient. While registration does not significantly add to the processing time of the pipeline overall, it nevertheless introduces an additional source of error and variability. In healthy data, registration tools are largely robust, however in some subjects, including those featuring pathology or who were scanned with a non-standard head orientation (as in many intraoperative cases), registration can prove less robust, required manual intervention and parameter tweaking.

Finally, tractfinder relies on HARDI diffusion data, and thus does not benefit from the shorter scan-times afforded by the simpler diffusion tensor acquisitions which are sufficient for the diffusion tensor-based tractography tools available on commercial neuronavigation tools.

### ROI Definitions

The following ROI strategies were used for atlas constructions and subsequent validation tractography (differences between the two specified where applicable).

### Arcuate Fasciculus:

Seed: White matter medial of angular gyrus, visible on coronal views of colour fractional anisotropy maps as a “green triangle”, drawn on the coronal plane. Level of coronal plane selected from sagittal view by locating the central sulcus.

Include: Descending section of the arcuate fasciculus, drawn on the axial plane

Exclude: Exclusion ROIs targeting: midline, superior fronto-occipital fasciculus, ipsilateral cerebral penduncles, sagittal stratum, corona radiata and external capsules.

Corticospinal tract: Corticospinal tract tracography strategy differed between the atlas creation and general tractography applied to new subjects.

Seed (atlas): For the orientation atlas, Freesurfer cortical parcellations were used to obtain more complete coverage of the motor cortex via the following process:
1. Seed in precentral gyrus and output successful seed location
2. Generate binary mask from successful seed locations, subtract from precentral gyrus mask to create seed mask
3. Re-run tractography with second seed mask to cover rest of precentral gyrus

Seed (general): Posterior limb of internal capsule, drawn on 3 consecutive axial slices

Include: Posterior limb on internal capsule (if not used for seed), cerebral penduncles, CST in mid-pons

Exclude: Cerebellar peduncles (drawn on coronal slice), medial lemniscus (drawn on axial slice), midline, superior fronto-occipital fasciculus,

### Optic Radiation:

Seed: Lateral geniculate nucleus (LGN; drawn on axial planes)

Include: Sagittal stratum (drawn on coronal plane)

Exclude: Coronal slice anterior of and axial slice inferior of most anterior point of lateral ventricles, axial slice at level of superior reach lateral ventricles, splenium of corpus callosum, fornix

Tracking parameters: Default parameters as documented for the tckgen command of MRtrix3 (release version 3.0.3, available at https://mrtrix.readthedocs.io/en/3.0.3/reference/commands/tckgen.html) (including—select 5000—algorithm iFOD2) were used for all tractography. In addition, the parameter—seed_unidirectional was included for optic radiation reconstructions, to ensure streamlines are propagated from a single direction out of the LGN.

### Notes on Tract Definitions

Corticospinal tract: Standardised white matter atlases and tractography protocols varyingly describe the corticospinal and pyramidal tracts. These two terms are often used interchangeably in tractography-oriented publications, while in anatomical terms they are distinct: The corticospinal (CST) and pyramidal tracts (PyT) are both descending motor pathways, with the PyT encompassing both the CST and the corticobulbar tract, which controls movement of the head, neck and face via the cranial nerves. Tractography studies and related white matter segmentation research frequently conflate the major descending (motor) and ascending (sensory pathways). This is evident in two main regions. Firstly, the inclusion of the medial lemniscus is frequently seen in PyT or CST segmentations (usually as it is not explicitly excluded, rather than being actively included). This includes TractSeg (and associated reference streamline bundles), XTRACT to some extent, and TractoInferno. By contrast, the tractography protocol employed in this research includes an exclusion mask on the medial lemniscus.

Secondly, while it has been suggested that the primary motor cortex can reside in the post-central gyrus, it is generally accepted that the somatosensory cortex is located in the latter, while the motor areas are in the precentral gyri. However, particularly with probabilistic tractography, it is near impossible to constrain streamlines exiting the internal capsule into the fanning corona radiata to one side of the central sulcus, without additional exclusion planes or the use of cortical target regions, which are especially time-consuming to produce, whether manually or through automatic parcellation. Thus streamline-based CST segmentations often contain parts of the somatosensory cortex while others, such as those utilising cortical parcellation-derived target regions, will be restricted to the motor cortex.

The tractfinder CST atlas streamlines were obtained using Freesurfer parcellations, as described in: “An automated labelling system for subdividing the human cerebral cortex on MRI scans into gyral based regions of interest” by Desikan et al. published in NeuroImage 31, 968{980. doi:DOI:10.1016/j.neuroimage.2006.01.021 in 2006, and also described in: “Whole brain segmentation: automated labeling of neuroanatomical structures in the human brain” by Fischl et al. published in Neuron 33, 341-355 in 2002, of the primary motor cortex, as are the TractSeg reference bundles. TractInferno reference bundles for the pyramidal tracts include sensory cortex.

Optic radiation: When it comes to the course of the optic radiations through the sagittal stratum and posterior termination in the occipital lobes, there is no disagreement between segmentation approaches. However, there remain significant differences in the regions of the lateral geniculate nucleus (LGN) and Meyer's loop. The LGN is a small nucleus of the thalamus from which the neurons of the OR originate. Its localisation on MRI images is not straightforward, and due to the complex arrangement of white matter structures in the upper midbrain and thalamus regions, it is easy for streamlines to extend into the entire posterior thalamus and fornix and even descend into the brainstem. This contributes to often broad OR segmentations in the thalamic portion at the start of the tract. Secondly, the full anterior extent of Meyer's loop is often not reconstructed by tractography, due to the extreme and tight curvature.

Arcuate fasciculus: Of the three tracts studied in this work, the arcuate fasciculus exhibits the most extreme variability in segmented anatomical extent. This is partially owing to disagreements in definition. For example, while the general consensus is that the arcuate fasciculus connects the temporal and frontal language areas, XTRACT follows the “three part” paradigm which includes a third cortical termination region in the supramarginal gyrus, or inferior parietal cortex. Furthermore, unless cortical parcellation derived termination masks are utilised, it is practically impossible to constrain streamlines to a compact pathway, with bundles frequently terminating within large swathes of the frontal and temporal lobes.

Those skilled in the art will appreciate that while the foregoing has described what is considered to be the best mode and where appropriate other modes of performing present techniques, the present techniques should not be limited to the specific configurations and methods disclosed in this description of the preferred embodiment. Those skilled in the art will recognise that present techniques have a broad range of applications, and that the embodiments may take a wide range of modifications without departing from any inventive concept as defined in the appended claims.

1. A computer-implemented method for imaging brain fibre tracts of a subject, the method comprising:
obtaining at least one tract-specific atlas of a brain, the atlas comprising a plurality of voxels indicating an expected location and a distribution of expected voxel-wise orientations of a specific brain fibre tract in a brain;
obtaining a diffusion magnetic resonance imaging, dMRI, image of a brain of the subject;
comparing the at least one obtained atlas with the obtained image to determine whether a brain fibre tract in the brain of the subject overlaps with the brain fibre tract of the atlas; and
generating, using the comparing, a modified image of the brain of the subject showing a location of the specific brain fibre tract in the brain of the subject.

2. The method as claimed in claim 1 wherein the method and obtaining an image of the brain of the subject is performed pre-surgery and/or during surgery.

3. (canceled)

4. The method as claimed in claim 1 further comprising:
modelling, using the obtained dMRI image, an orientation distribution of at least one brain fibre tract in each voxel of the image,
wherein the modelling comprises using any one of the following to determine an orientation distribution of at least one brain fibre tract in each voxel of the image: constrained spherical deconvolution: a multi-compartment model, a multi-tensor model; a multi-fibre model; and a ball-and-stick model.

5. (canceled)

6. The method as claimed in claim 1 wherein comparing the at least one obtained atlas with the obtained image comprises:
comparing each voxel of the at least one obtained atlas with each voxel of the obtained image.

7. The method as claimed in claim 6 wherein each voxel of the at least one obtained atlas comprises a distribution of expected orientations of the specific brain fibre tract that is expected to be located in the voxel, and wherein comparing each voxel comprises:
obtaining a measure per voxel of how closely an orientation distribution of a brain fibre tract in each voxel of the image overlaps with the distribution of expected orientations of the specific brain fibre tract.

8. The method as claimed in claim 6 wherein obtaining at least one tract-specific atlas of a brain comprises obtaining at least two tract-specific atlases, and when the at least two atlases indicate that two or more brain fibre tracts are likely to be located in a particular voxel, comparing each voxel comprises:
obtaining a measure per voxel of how closely an orientation distribution of a brain fibre tract in each voxel of the image overlaps with each distribution of expected orientations of the brain fibre tracts in the at least two atlases; and
determining which one of the two or more brain fibre tracts is present in the voxel of the obtained image based on the obtained measure.

9. The method as claimed in claim 6 wherein the atlas is represented by a first spherical distribution function, and the obtained image is represented by a second spherical distribution function, and wherein the comparing comprises calculating a voxel-wise integral of a product of the first and second functions.

10. (canceled)

11. The method as claimed in claim 6 wherein the atlas is represented by a first spherical distribution function, and the obtained image is represented by a second spherical distribution function, and wherein the calculating comprises calculating a Kullback-Leibler divergence metric using the first and second functions.

12. The method as claimed in claim 9 wherein generating a modified image comprises outputting an image representing a result of the calculating.

13. The method as claimed in claim 1 wherein obtaining the at least one atlas comprises obtaining at least one atlas indicating an expected location and an expected orientation of a specific brain fibre tract in a structurally normal brain.

14. The method as claimed in claim 1 wherein obtaining the at least one atlas comprises obtaining at least one atlas that has been pre-deformed by transforming an atlas indicating an expected location and an expected orientation of a specific brain fibre tract in a structurally normal brain using a tumour model that defines how brain fibre tracts are displaced by tumours, the pre-deformed atlas indicating an expected location and an expected orientation of a specific brain fibre tract in a brain containing a tumour.

15. (canceled)

16. A computer-implemented method for generating a tract-specific atlas of a structurally normal brain for use in brain fibre tract imaging, the method comprising:
obtaining a plurality of images of structurally normal brains of multiple subjects;
extracting, from each image, spatial location and orientation information of at least one brain fibre tract in the brain; and
generating, using the extracted spatial location and orientation information, an atlas comprising a plurality of voxels indicating an expected location and a distribution of expected orientations of at least one specific brain fibre tract.

17. The method as claimed in claim 16 wherein generating an atlas comprises:
determining, using the extracted spatial location information from the plurality of images, a likelihood of a specific brain fibre tract being located in a particular voxel; and/or
determining, using the extracted orientation information from the plurality of images, a distribution of orientations of a specific brain fibre tract in a particular voxel.

18. (canceled)

19. The method as claimed in claim 17 wherein, when two or more brain fibre tracts are likely to be located in a particular voxel, generating an atlas comprises:
including, in the particular voxel of the atlas, the distribution of expected orientations of each of the two or more brain fibre tracts.

20. The method as claimed in claim 17 where obtaining a plurality of images of brains comprises obtaining images acquired from a high angular resolution diffusion imaging, HARDI, process.

21. A computer-implemented method for generating a pre-deformed atlas for use in brain fibre tract imaging, the method comprising:
obtaining information about a subject having a brain tumour;
obtaining a tract-specific atlas of a specific brain fibre tract of interest, the atlas indicating an expected location and an expected orientation of the specific brain fibre tract in a structurally normal brain;
transforming the atlas, using the obtained information and a tumour model that defines how brain fibre tracts are displaced by tumours, to generate a pre-deformed atlas.

22. The method as claimed in claim 21 wherein obtaining information about a subject having a brain tumour comprises obtaining information on a location of the brain tumour acquired from an image of the brain of the subject.

23. (canceled)

24. The method as claimed in claim 21 wherein the tumour model is a radial tumour expansion model.

25. The method as claimed in claim 21 wherein the atlas comprises a plurality of voxels, and transforming the atlas comprises:
defining, for each voxel, a distance to a centre of mass of a tumour; and
applying, to each voxel, a function which defines an amount by which each voxel is displaced as being dependent on the distance from the voxel to a centre of mass of a tumour, a distance from the centre of mass to a surface of the brain, and a distance from the centre of mass to a surface of the tumour, wherein the function is any one of: an exponentially decaying function: a polynomial function: a probability density function of a logistic distribution or of a hyperbolic secant distribution function: a damped oscillator function; and a linear function.

26. (canceled)

27. The method as claimed in claim 21 wherein the tumour model models how fibre tracts are displaced by infiltrating and/or non-infiltrating tumours.

28. (canceled)

29. (canceled)

30. (canceled)

31. (canceled)
